SEQUENCE LISTING (15 sequences)

SEQ ID NO: 1 - DNA, 12361 bp, synthetic construct
  source 1..12361: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaagttcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctcctcaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttctcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccctatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatcgatggcgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggctagcatcgagggccggggcggctggggcaaagagacagccgccgccaagttcgagcggcagcacatggacagctgataattaattaagtaaccgatacagcagcaattggcaagctgcttacatagaactcgcggcgattggcatgccgctttaaaatttttattttattttttcttttcttttccgaatcggattttgtttttaatatttcaaaaaaaaaaaaaaaaaaaaaaaaaaaaaaaaaaaaaaaaaaaaaaaaaaggaagagcgcggccgcatccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatttctagaggatccctaatacgactcactatag

SEQ ID NO: 2 - DNA, 4003 bp, synthetic construct
  source 1..4003: mol_type = other DNA
ttaattaagtaaccgatacagcagcaattggcaagctgcttacatagaactcgcggcgattggcatgccgctttaaaatttttattttattttttcttttcttttccgaatcggattttgtttttaatatttcaaaaaaaaaaaaaaaaaaaaaaaaaaaaaaaaaaaaaaaaaaaaaaaaaaggaagagcgcggccgcatccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatttctagaggatccctaatacgactcactatag

SEQ ID NO: 3 - DNA, 7560 bp, synthetic construct
  source 1..7560: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaagttcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctcctcaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttctcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccctatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatcgatggcgcgccacc

SEQ ID NO: 4 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 5 - DNA, 7691 bp, synthetic construct
  source 1..7691: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcggccgcatgagtctggccattccagttatgtgcctgctggcaaataccacgttcccctgctcccagcccccttgcacaccctgctgctacgaaaaagagccggagaaaaccttgcgcatgcttgaagacaatgtcatgagccccgggtactatcagctgctacaagcatccttaacatgttctccccgacgccagcggcgcagtattaaggaccacttcaatgtctataaagccacaagaccgtacctagctcactgtcccgactgtggagaagggcactcgtgccatagtcccgtagcgctagaacgcatcagaaacgaagcgacagacgggacgttgaaaatccaggtttccttgcaaatcggaataaagacggatgatagccatgattggaccaagctgcgttatatggacaatcacatgccagcagacgcagagcgggccgggctatttgtaagaacgtcagcaccgtgcacgattactggaacaatgggacacttcattctggcccgatgtccgaaaggagaaactctgacggtagggttcactgacggtaggaagatcagtcactcatgtacgcacccatttcaccatgaccctcctgtgataggccgggaaaaattccattcccgaccgcagcacggtagggaactaccttgcagcacgtacgcgcagagcaccgctgcaactgccgaggagatagaggtacatatgcccccagacaccccagatcgcacattaatgtcacaacagtccggcaatgtaaagatcacagtcaatagtcagacggtgcggtacaagtgcaattgtggtgactcaagtgaaggattaaccactacagataaagtgattaataactgcaaggttgatcaatgccatgccgcggtcaccaatcacaaaaaatggcagtataattcccctctggtcccgcgtaatgctgaattcggggaccggaaaggaaaagttcacattccatttcctctggcaaatgtgacatgcagggtgcctaaagcaagaaaccccaccgtgacgtacggaaaaaaccaagtcatcatgttgctgtatcctgaccacccaacgctcctgtcctacaggaatatgggagaagaaccaaactatcaagaagagtgggtgacgcataagaaggagatcaggttaaccgtgccgactgaagggctcgaggtcacgtggggtaacaatgagccgtacaagtattggccgcagttatccacaaacggtacagcccacggccacccgcatgagataattctgtattattatgagctgtacccaactatgactgtggtagttttgtcagtggcctcgttcatactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaggaggccgcggtatacctgtggaacgagcagcagcctttattttggctgcaagcccttattccgctggcagccctgattgtcctatgtaactgtctgagactcttaccatgctgttgtaaaatgttgacttttttagccgtactgagcgtcggtgcccacactgtgagtgcgtacgaacacgtaacagtgatcccgaacacggtgggagtaccgtataagactctagtcaacagaccgggctacagccccatggtattggagatggagcttctgtctgtcaccttggaaccaacgctatcgcttgattacatcacgtgcgagtataaaaccgttatcccgtctccgtacgtgaaatgctgcggtacagcagagtgtaaggacaagagcctacctgattacagctgtaaggtcttcaccggcgtctacccattcatgtggggcggcgcctactgcttctgcgacaccgaaaatacgcaattgagcgaagcacatgtggagaagtccgaatcatgcaaaacagaatttgcatcagcatacagggctcataccgcatccgcgtcagctaagctccgcgtcctttaccaaggaaataatatcactgtagctgcttatgcaaacggcgaccatgccgtcacagttaaggacgctaaattcatagtggggccaatgtcttcagcctggacacctttcgacaataaaatcgtggtgtacaaaggcgacgtctacaacatggactacccgcccttcggcgcaggaagaccaggacaatttggcgacatccaaagtcgcacgcctgagagcgaagacgtctatgctaatacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggcctttaccagggtcgtcgacgcgccatctttaacggacatgtcgtgtgaggtatcagcctgcacccattcctcagactttgggggcgtagccatcattaaatatgcagccagtaagaaaggcaagtgtgcagtgcactcgatgactaacgccgtcactattcgggaagctgaaatagaagtagaagggaactctcagttgcaaatctctttttcgacggccctagccagcgccgaatttcgcgtacaagtctgttctacacaagtacactgtgcagccgagtgccatccaccgaaagaccatatagtcaattacccggcgtcacacaccaccctcggggtccaagacatttccgctacggcgatgtcatgggtgcagaagatcacgggaggtgtgggactggttgtcgctgttgcagcactgatcctaatcgtggtgctatgcgtgtcgtttagcaggcactaagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 6 - DNA, 4724 bp, synthetic construct
  source 1..4724: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcggccgcgctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 7 - DNA, 2967 bp, synthetic construct
  source 1..2967: mol_type = other DNA
atgagtctggccattccagttatgtgcctgctggcaaataccacgttcccctgctcccagcccccttgcacaccctgctgctacgaaaaagagccggagaaaaccttgcgcatgcttgaagacaatgtcatgagccccgggtactatcagctgctacaagcatccttaacatgttctccccgacgccagcggcgcagtattaaggaccacttcaatgtctataaagccacaagaccgtacctagctcactgtcccgactgtggagaagggcactcgtgccatagtcccgtagcgctagaacgcatcagaaacgaagcgacagacgggacgttgaaaatccaggtttccttgcaaatcggaataaagacggatgatagccatgattggaccaagctgcgttatatggacaatcacatgccagcagacgcagagcgggccgggctatttgtaagaacgtcagcaccgtgcacgattactggaacaatgggacacttcattctggcccgatgtccgaaaggagaaactctgacggtagggttcactgacggtaggaagatcagtcactcatgtacgcacccatttcaccatgaccctcctgtgataggccgggaaaaattccattcccgaccgcagcacggtagggaactaccttgcagcacgtacgcgcagagcaccgctgcaactgccgaggagatagaggtacatatgcccccagacaccccagatcgcacattaatgtcacaacagtccggcaatgtaaagatcacagtcaatagtcagacggtgcggtacaagtgcaattgtggtgactcaagtgaaggattaaccactacagataaagtgattaataactgcaaggttgatcaatgccatgccgcggtcaccaatcacaaaaaatggcagtataattcccctctggtcccgcgtaatgctgaattcggggaccggaaaggaaaagttcacattccatttcctctggcaaatgtgacatgcagggtgcctaaagcaagaaaccccaccgtgacgtacggaaaaaaccaagtcatcatgttgctgtatcctgaccacccaacgctcctgtcctacaggaatatgggagaagaaccaaactatcaagaagagtgggtgacgcataagaaggagatcaggttaaccgtgccgactgaagggctcgaggtcacgtggggtaacaatgagccgtacaagtattggccgcagttatccacaaacggtacagcccacggccacccgcatgagataattctgtattattatgagctgtacccaactatgactgtggtagttttgtcagtggcctcgttcatactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaggaggccgcggtatacctgtggaacgagcagcagcctttattttggctgcaagcccttattccgctggcagccctgattgtcctatgtaactgtctgagactcttaccatgctgttgtaaaatgttgacttttttagccgtactgagcgtcggtgcccacactgtgagtgcgtacgaacacgtaacagtgatcccgaacacggtgggagtaccgtataagactctagtcaacagaccgggctacagccccatggtattggagatggagcttctgtctgtcaccttggaaccaacgctatcgcttgattacatcacgtgcgagtataaaaccgttatcccgtctccgtacgtgaaatgctgcggtacagcagagtgtaaggacaagagcctacctgattacagctgtaaggtcttcaccggcgtctacccattcatgtggggcggcgcctactgcttctgcgacaccgaaaatacgcaattgagcgaagcacatgtggagaagtccgaatcatgcaaaacagaatttgcatcagcatacagggctcataccgcatccgcgtcagctaagctccgcgtcctttaccaaggaaataatatcactgtagctgcttatgcaaacggcgaccatgccgtcacagttaaggacgctaaattcatagtggggccaatgtcttcagcctggacacctttcgacaataaaatcgtggtgtacaaaggcgacgtctacaacatggactacccgcccttcggcgcaggaagaccaggacaatttggcgacatccaaagtcgcacgcctgagagcgaagacgtctatgctaatacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggcctttaccagggtcgtcgacgcgccatctttaacggacatgtcgtgtgaggtatcagcctgcacccattcctcagactttgggggcgtagccatcattaaatatgcagccagtaagaaaggcaagtgtgcagtgcactcgatgactaacgccgtcactattcgggaagctgaaatagaagtagaagggaactctcagttgcaaatctctttttcgacggccctagccagcgccgaatttcgcgtacaagtctgttctacacaagtacactgtgcagccgagtgccatccaccgaaagaccatatagtcaattacccggcgtcacacaccaccctcggggtccaagacatttccgctacggcgatgtcatgggtgcagaagatcacgggaggtgtgggactggttgtcgctgttgcagcactgatcctaatcgtggtgctatgcgtgtcgtttagcaggcactaa

SEQ ID NO: 8 - DNA, 2946 bp, synthetic construct
  source 1..2946: mol_type = other DNA
atgtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacaataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaaccacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcatatatccacccggagttcaggctccaaatatgcacatcatatgttacgtgcaaaggtgattgtcaccccccgaaagaccattttgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattga

SEQ ID NO: 9 - DNA, 2039 bp, synthetic construct
  source 1..2039: mol_type = other DNA
atgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttagttaattaa

SEQ ID NO: 10 - DNA, 3532 bp, synthetic construct
  source 1..3532: mol_type = other DNA
atgccggcagaaaacaagaaagttagattcgaaaatactacttcagacaaagggaaaattcctagtaaagttattaagagctactacggaaccatggacattaagaaaataaatgaaggattattggacagcaaaatattaagtgctttcaacacagtaatagcattgcttggatctatcgtgatcatagtgatgaatataatgatcatccaaaattacacaagatcaacagacaatcaggccgtgatcaaagatgcgttgcagggtatccaacagcagatcaaagggcttgctgacaaaatcggcacagagatagggcccaaagtatcactgattgacacatccagtaccattactatcccagctaacattgggctgttaggttcaaagatcagccagtcgactgcaagtataaatgagaatgtgaatgaaaaatgcaaattcacactgcctcccttgaaaatccacgaatgtaacatttcttgtcctaacccactcccttttagagagtataggccacagacagaaggggtgagcaatctagtaggattacctaataatatttgcctgcaaaagacatctaatcagatattgaagccaaagctgatttcatacactttacccgtagtcggtcaaagtggtacctgtatcacagacccattgctggctatggacgagggctattttgcatatagccacctggaaagaatcggatcatgttcaagaggggtctccaaacaaagaataataggagttggagaggtactagacagaggtgatgaagttccttctttatttatgaccaatgtctggaccccaccaaatccaaacaccgtttaccactgtagtgctgtatacaacaatgaattctattatgtactttgtgcagtgtcaactgttggagaccctattctgaatagcacctactggtccggatctctaatgatgacccgtctagctgtgaaacccaagagtaatggtgggggttacaatcaacatcaacttgccctacgaagtatcgagaaagggaggtatgataaagttatgccgtatggaccttcaggcatcaaacagggtgacaccctgtattttcctgctgtaggatttttggtcaggacagagtttaaatacaatgattcaaattgtcccatcacgaagtgtcaatacagtaaacctgaaaattgcaggctatctatggggattagaccaaacagccattatatccttcgatctggactattaaaatacaatctatcagatggggagaaccccaaagttgtattcattgaaatatctgatcaaagattatctattggatctcctagcaaaatctatgattctttgggtcaacctgttttctaccaagcgtcattttcatgggatactatgattaaatttggagatgttctaacagtcaaccctctggttgtcaattggcgtaataacacggtaatatcaagacccgggcaatcacaatgccctagattcaatacatgtccagagatctgctgggaaggagtttataatgatgcattcctaattgacagaatcaattggataagcgcgggtgtattccttgacagcaatcagaccgcagaaaatcctgtttttactgtattcaaagataatgaaatactttatagggcacaactggcttctgaggacaccaatgcacaaaaaacaataactaattgttttctcttgaagaataagatttggtgcatatcattggttgagatatatgacacaggagacaatgtcataagacccaaactattcgcggttaagataccagagcaatgtacataattaattaatacctgagaggggcccctataactctctacggctaacctgaatggactacgacatagtctagtccgccaagaccatggtagttatacttgacaagagatgttattgtaatcttttaatattgattttgatgatctcggagtgtagtgttgggattctacattatgagaaattgagtaaaattggacttgtcaaaggagtaacaagaaaatacaagattaaaagcaatcctctcacaaaagacattgttataaaaatgattccgaatgtgtcgaacatgtctcagtgcacagggagtgtcatggaaaattataaaacacgattaaacggtatcttaacacctataaagggagcgttagagatctacaaaaacaacactcatgaccttgtcggtgatgtgagattagccggagttataatggcaggagttgctattgggattgcaaccgcagctcaaatcactgcaggtgtagcactatatgaggcaatgaagaatgctgacaacatcaacaaactcaaaagcagcattgaatcaactaatgaagctgtcgttaaacttcaagagactgcagaaaagacagtctatgtgctgactgctctacaggattacattaatactaatttagtaccgacaattgacaagataagctgcaaacagacagaactctcactagatctggcattatcaaagtacctctctgatttgctttttgtatttggccccaaccttcaagacccagtttctaattcaatgactatacaggctatatctcaggcattcggtggaaattatgaaacactgctaagaacattgggttacgctacagaagactttgatgatcttctagaaagtgacagcataacaggtcaaatcatctatgttgatctaagtagctactatataattgtcagggtttattttcctattctgactgaaattcaacaggcctatatccaagagttgttaccagtgagcttcaacaatgataattcagaatggatcagtattgtcccaaatttcatattggtaaggaatacattaatatcaaatatagagattggattttgcctaattacaaagaggagcgtgatctgcaaccaagattatgccacacctatgaccaacaacatgagagaatgtttaacgggatcgactgagaagtgtcctcgagagctggttgtttcatcacatgttcccagatttgcactatctaacggggttctgtttgccaattgcataagtgttacatgtcagtgtcaaacaacaggcagggcaatctcacaatcaggagaacaaactctgctgatgattgacaacaccacctgtcctacagccgtactcggtaatgtgattatcagcttagggaaatatctggggtcagtaaattataattctgaaggcattgctatcggtcctccagtctttacagataaagttgatatatcaagtcagatatccagcatgaatcagtccttacaacagtctaaggactatatcaaagaggctcaacgactccttgatactgttaatccatcattaataagcatgttgtctatgatcatactgtatgtattatcgatcgcatcgttgtgtatagggttgattacatttatcagttttatcattgttgagaaaaagagaaacacctacagcagattagaggataggagagtcagacctacaagcagtggggatctctactacattgggacatag

SEQ ID NO: 11 - DNA, 1476 bp, synthetic construct
  source 1..1476: mol_type = other DNA
atgggacaaatagtgacattcttccaggaagtgcctcatgtaatagaagaggtgatgaacattgttctcattgcactgtctgtactagcagtgctgaaaggtctgtacaattttgcaacgtgtggccttgttggtttggtcactttcctcctgttgtgtggtaggtcttgcacaaccagtctttataaaggggtttatgagcttcagactctggaactaaacatggagacactcaatatgaccatgcctctctcctgcacaaagaacaacagtcatcattatataatggtgggcaatgagacaggactagaactgaccttgaccaacacgagcattattaatcacaaattttgcaatctgtctgatgcccacaaaaagaacctctatgaccacgctcttatgagcataatctcaactttccacttgtccatccccaacttcaatcagtatgaggcaatgagctgcgattttaatgggggaaagattagtgtgcagtacaacctgagtcacagctatgctggggatgcagccaaccattgtggtactgttgcaaatggtgtgttacagacttttatgaggatggcttggggtgggagctacattgctcttgactcaggccgtggcaactgggactgtattatgactagttatcaatatctgataatccaaaatacaacctgggaagatcactgccaattctcgagaccatctcccatcggttatctcgggctcctctcacaaaggactagagatatttatattagtagaagattgctaggcacattcacatggacactgtcagattctgaaggtaaagacacaccagggggatattgtctgaccaggtggatgctaattgaggctgaactaaaatgcttcgggaacacagctgtggcaaaatgtaatgagaagcatgatgaggaattttgtgacatgctgaggctgtttgacttcaacaaacaagccattcaaaggttgaaagctgaagcacaaatgagcattcagttgatcaacaaagcagtaaatgctttgataaatgaccaacttataatgaagaaccatctacgggacatcatgggaattccatactgtaattacagcaagtattggtacctcaaccacacaactactgggagaacatcactgcccaaatgttggcttgtatcaaatggttcatacttgaacgagacccacttttctgatgatattgaacaacaagctgacaatatgatcactgagatgttacagaaggagtatatggagaggcaggggaagacaccattgggtctagttgacctctttgtgttcagtacaagtttctatcttattagcatcttccttcacctagtcaaaataccaactcataggcatattgtaggcaagtcgtgtcccaaacctcacagattgaatcatatgggcatttgttcctgtggactctacaaacagcctggtgtgcctgtgaaatggaagagatga

SEQ ID NO: 12 - DNA, 1572 bp, synthetic construct
  source 1..1572: mol_type = other DNA
atggttcctcaggctctcctgtttgtaccccttctggtttttccattgtgttttgggaaattccctatttacacgataccagacaagcttggtccctggagtccgattgacatacatcacctcagctgcccaaacaatttggtagtggaggacgaaggatgcaccaacctgtcagggttctcctacatggaacttaaagttggatacatcttagccataaaagtgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttacctccagtagagggaagagagcatccaaagggagtgagacttgcggctttgtagatgaaagaggcctatataagtctttaaaaggagcatgcaaactcaagttatgtggagttctaggacttagacttatggatggaacatgggtctcgatgcaaacatcaaatgaaaccaaatggtgccctcccgataagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcgaaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactg

SEQ ID NO: 13 - DNA, 1350 bp, synthetic construct
  source 1..1350: mol_type = other DNA
atgaatccaaatcagaagatagtgaccattggatcaatctgtatggtaagtgggataattagcttgatgttacagattgggaacataatctcaatatgggtcagtcattcaattcaaacagggaatcaacaccaaactgaaccaatcagaaatactaatttcctcactgagaacactgtagcttcagtaacattagctggcaattcatctctttgccccatcaaaggatgggctgtacacagtaaagacaacagtataaggattgggtccaagggggatgtgtttgtaattagagagccgttcatatcatgctcccatctggaatgcagaaccttctttttgactcagggagctttactgaatgacaagcactccaacgggactttcaaagataggagccctcatagaacgctaatgagttgccctataggtgaggctccctctccatataactcgaggtttgagtctgttgcttggtcggcaagtgcttgccatgatggcaccagttggttgataattggaatttctggtccagacaatggggctgtggcggtattgaaatacaatggcataataacagacactatcaagagttggaggaataacatactgaggacccaagagtctgaatgtgcatgtatgaatggctcttgctttactgttatgacagatggaccaagtaatgggcaggcatcatataagattttcaaaatagaaaaagggaaagtagttaagtcagtcgaattgaatgcccccaattatcactatgaggaatgctcctgttatcctgaggctggcgaaatcatatgtgtgtgcagggataattggcatggctcaaacaggccatgggtatctttcaatcagaatttggagtatcaaataggatatatttgcagtggagttttcggtgacaatccacggccgaatgacggtacagggagttgtggaccagtgtcctctaacggggcatatggggtaaaagggttctcatttaaatatggcaatggtgtctggatcgggaggaccaaaagcactcattccaggagcggctttgaaatgatttgggatccaaacgggtggactggaacggacagtgagttctcgatgaaacaagatatagtagcaataactgattggtcaggatatagcgggagttttgtccagcacccagaactgacaggattggattgcatgagaccttgcttctgggttgagttaatcagagggcggcccaaagagagcacaatttggactagtgggagcagcatatctttttgtggtgtaaatagtgacactgtgagttggtcttggccagacggtgctgagttgccattcaccattgacaagtag

SEQ ID NO: 14 - DNA, 1725 bp, synthetic construct
  source 1..1725: mol_type = other DNA
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 15 - DNA, 2415 bp, synthetic construct
  source 1..2415: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttt

(19) United States
(12) Patent Application Publication
(10) Pub. No.: US 2026/0110000 A1
(43) Pub. Date: Apr. 23, 2026

(54) SARNA DELIVERY SYSTEM, PREPARATION METHOD THEREFOR AND USE THEREOF

(71) Applicant: ZHEJIANG FREE TRADE ZONE HONGAN BASE BIOTECHNOLOGY CO., LTD., Zhoushan (CN)

(72) Inventors: Bo ZHANG, Zhoushan (CN); Junsheng LIU, Zhoushan (CN); Shunli ZHAN, Zhoushan (CN)

(73) Assignee: ZHEJIANG FREE TRADE ZONE HONGAN BASE BIOTECHNOLOGY CO., LTD., Zhoushan (CN)

(21) Appl. No.: 19/356,226

(22) Filed: Oct. 13, 2025

Related U.S. Application Data
(63) Continuation of application No. PCT/CN2024/087266, filed on Apr. 11, 2024.

(30) Foreign Application Priority Data
Apr. 11, 2023 (CN) 202310381377.5

(51) Int. Cl. C12N 15/88; C12N 5/071; C12N 15/113

(52) U.S. Cl. CPC C12N 15/88; C12N 5/0686; C12N 15/113

(57) ABSTRACT

A method for constructing a saRNA microvesicle delivery system includes the following steps: (1) designing a saRNA expression vector 1 and a membrane protein expression vector 2; and (2) co-transfecting the expression vector 1 and the expression vector 2 into a host cell; and (3) culturing the host cell, and isolating microvesicles containing a saRNA, the saRNA containing a target gene sequence, a replicon in the saRNA expression vector being an alphavirus replicon, and the microvesicles not containing capsid protein. The system can efficiently deliver the saRNA to cells or in vivo to realize the expression of a target protein. On this basis, different elements in the delivery system can be further transformed to develop a high-efficiency low-immunogenicity modular platform aiming at different diseases and different targets. The delivery system may supplement the toolbox of existing viral and lipid nanoparticle delivery vectors and has good application prospects.

### CROSS REFERENCE TO THE RELATED APPLICATIONS

This application is a continuation application of International Application No. PCT/CN2024/087266, filed on Apr. 11, 2024, which is based upon and claims priority to Chinese Patent Application No. 202310381377.5, filed on Apr. 11, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBZD046_SequenceListing.xml, created on 10/11/2025, and is 70,980 bytes in size.

### TECHNICAL FIELD

The present disclosure belongs to the field of biotechnology, particularly relates to a novel self-amplifying RNA (saRNA) delivery system, which mediates saRNA delivery via microvesicles assembled from viral envelope proteins (E), and achieves targeted expression of target proteins through the self-replication characteristic of saRNA.

### BACKGROUND

After years of technological accumulation, the gene therapy industry has gradually matured, leading the third industrial revolution in biomedicine. Among them, mRNA drugs, as relatively safe preparations, have attracted the attention of researchers both domestically and internationally. They achieve therapeutic purposes by stimulating the immune system to respond or expressing and producing corresponding proteins. Therefore, the expression level of the downstream proteins is closely related to the therapeutic efficacy. One of the drawbacks of traditional mRNA is its instability and susceptibility to degradation, resulting in non-sustained downstream protein expression. If used long-term for disease treatment, patients may need to be injected with large amounts of mRNA preparations, which may increase the toxic and side effects of mRNA therapy. Self-amplifying RNA (saRNA) refers to a nucleic acid sequence that uses its own RNA sequence as a template and undergoes self-replication with the assistance of the RNA polymerase (RdRP) it carries. The primary advantage of saRNA is that it can achieve the same protein expression level as a large amount of traditional mRNA at a very low dose and can continuously produce the target protein. This characteristic can reduce the injection dose and frequency in RNA preparation therapy, while prolonging the therapeutic effect and at the same time reducing potential toxic and side effects that may be caused by RNA and administration vectors.

In addition, the efficient delivery of mRNA preparations has always been one of the important bottlenecks hindering the development of gene drug and gene therapy industries. At present, delivery vectors used in human gene therapy mainly include non-viral vectors such as liposomes, cationic polymers, nanoparticles, etc., and viral vectors such as lentiviruses, adenoviruses and adeno-associated viruses. Non-viral delivery vectors have the characteristics of safety, stability, etc., but they usually have low transfection efficiency, high production costs and obvious side effects. Viral delivery vectors, including two categories, i.e., live viruses and pseudoviruses, are widely used due to their high transduction and expression efficiency. However, live viral vectors, such as influenza viruses, measles viruses, Newcastle disease viruses, and Virus-like vesicles (VLVs), etc., can produce progeny viruses in vivo, leading to potential infection risks. They are usually only suitable for gene delivery in the field of vaccines, not for protein drugs, cell and gene therapy, etc., which restricts the development of such vectors in gene delivery. In comparison, pseudoviral vectors have the advantages of only being singly infectious, having no toxic or side effects caused by viral amplification, etc. Their extremely high safety makes pseudoviral vectors have great developmental and application potential as RNA delivery tools, among which the most representative is Virus-replicon particles (VRPs). The VRP delivery system can effectively deliver saRNA into the body through pseudoviral replicon particles with only single infectivity produced by the trans-complementation of alphavirus replicon RNA and alphavirus structural protein RNA. However, the delivery system in the form of VRP also has the fatal defect of generating wild-type viruses through genetic recombination. For example, the packaging system of Venezuelan equine encephalitis virus (VEEV)-based VRP consists of three parts: VEEV replicon RNA, capsid protein RNA, and E protein RNA. These RNAs have a low probability of recombination in cells, and may produce VEEV viruses. In order to minimize the recombination risk in the VRP delivery system, a three-plasmid system is usually employed to transcribe VEEV replicon RNA, capsid protein RNA, and E protein RNA, respectively. Even so, the possibility of generating VEEV viruses through recombination cannot be completely avoided. In addition, the VRP delivery system also has some other defects: due to the presence of capsid proteins, on one hand, it limits the exposure of saRNA, and on the other hand, it enhances the host's antiviral immune response, i.e., it not only affects the RNA delivery efficiency but also affects the side reactions during the drug use. Therefore, the VRP delivery system is usually only used for gene delivery in a few fields such as vaccines. For the above reasons, the entire biomedical field is striving to develop new and more potential gene delivery systems, but it is full of challenges to accurately and effectively deliver these molecules into cells while ensuring safety.

Viral E proteins can be assembled relying on negatively charged region and the signal peptide at their C-terminus to form the envelope structure of viral particles. During the viral life cycle, viral E proteins interact with other viral components, such as nucleoproteins, matrix proteins, etc., to package infectious viral particles. The process of viral replication and packaging to produce progeny viruses is completely dependent on the presence of viral structural proteins such as viral nucleoproteins, matrix proteins, etc. When the viral genome lacks structural genes such as nucleoproteins, matrix proteins, etc., it usually cannot produce progeny viral particles.

The interaction between RNA and proteins is one of the decisive factors for the realization of cellular physiological processes. In recent years, with the improvement of technologies and the establishment of new methods, significant progress has been made in the research on the interaction between RNA and proteins. At present, researchers have identified many protein-binding sites on RNA, discovered many RNA-binding domains in proteins, and conducted relatively detailed studies on their structural characteristics. Viral E protein is the main membrane protein on the viral envelope, which not only mediates the binding of the virus to cell receptors but also plays an important role in the formation of the viral envelope.

Therefore, how to explore the potential of related interaction between viral E proteins and mRNA, develop a new mRNA drug delivery system by utilizing the characteristics of viral E proteins in combination with the safety and efficiency of saRNA, and develop new and more mature mRNA drug preparations require further exploration by those skilled in the art.

### SUMMARY

To address the issues existing in the prior art, the present disclosure aims to provide a novel saRNA delivery (VRV) system and construction method therefor. By introducing saRNA sequences and viral envelope protein RNA sequences into cells via a two-plasmid transfection system, a cellular microvesicle delivery system containing saRNA can be effectively packaged, which is used for packaging and delivering saRNA, and achieving the expression of target proteins through saRNA. This new form of delivery system greatly expands the ways of gene drug delivery.

To achieve the above purposes, the technical solutions of the present disclosure are as follows:

In one aspect, the present disclosure provides a method for constructing a saRNA microvesicle delivery system, including the following steps:
(1) designing a saRNA expression vector 1 and a membrane protein expression vector 2;
(2) co-transfecting the expression vector 1 and the expression vector 2 into a host cell;
(3) culturing the host cell and isolating to obtain a microvesicle containing saRNA;
wherein, the saRNA includes a target gene sequence;
the replicon in the saRNA expression vector is an alphavirus replicon;
the microvesicle does not include a capsid protein.

In another aspect, the present disclosure provides a microvesicle delivery vector prepared by the aforementioned method, wherein the delivery vector includes a microvesicle with a viral membrane protein embedded on the surface and saRNA packaged in the vesicle, the microvesicle does not include a capsid protein; preferably, the saRNA also includes a target gene sequence.

In another aspect, the present disclosure provides a cell stably expressing the aforementioned microvesicle delivery vector.

In another aspect, the present disclosure provides a use of the aforementioned method, microvesicle delivery vector and/or cell in the manufacture of a targeted drug.

In another aspect, the present disclosure provides a use of the aforementioned method, microvesicle delivery vector and/or cell in the manufacture of a vaccine preparation.

The beneficial effects achieved by the present disclosure are at least as follows:

Through research on the interaction between saRNA and viral membrane proteins, the present disclosure has found that viral E proteins can interact with saRNA, and in mammalian cells, viral E proteins can efficiently mediate the production of cellular microvesicles containing saRNA. This interaction may depend on the specific transmembrane domain and protein's tertiary structure in the intracellular domain of the viral E protein, as well as the tertiary structure of saRNA. When viral membrane proteins are absent, although cells can also spontaneously produce extracellular vesicles, they cannot effectively package saRNA into the vesicles; at the same time, there are certain differences in the interaction effects between different viral E proteins and saRNA, and the efficiency of producing microvesicles packaging saRNA also varies. Optimizing the viral E protein RNA sequence and saRNA sequence can effectively promote the interaction between viral E proteins and saRNA, thereby significantly improving the production efficiency of microvesicles packaging saRNA.

The present disclosure enable the effective packaging of cellular microvesicles containing saRNA by introducing saRNA sequences and viral envelope protein RNA sequences into cells via a two-plasmid transfection system. This novel saRNA delivery (VRV) system mediated by viral envelope protein-assembled microvesicles can effectively mediate in vivo delivery of saRNA and has outstanding delivery advantages. The VRV delivery system differs from VLV in that it is not an active virus. Only the saRNA drug component is delivered into the body, without any viral structural protein genes, and it cannot produce progeny viruses. This overcomes the fatal drawbacks of viral vectors and ensures favorable safety in use. Secondly, the VRV delivery system is different from VRP: VRP is a virus-like particle of alphavirus, whose key mechanism of saRNA packaging lies in the interaction between saRNA and nucleoproteins. Therefore, viral nucleoprotein is crucial for the formation of VRP, and VRP includes a large amount of viral nucleoprotein components; VRV is a type of cellular microvesicles, whose key mechanism of saRNA packaging is the interaction between saRNA and viral membrane proteins. VRV does not include viral nucleoprotein components and has lower immunogenicity, making it more suitable for RNA delivery in medical fields such as protein replacement drugs, antibody drugs, cell and gene therapy drugs, etc. In addition, the VRV packaging system does not require the use of viral nucleoprotein RNA. The packaging of VRV can be completed using a two-plasmid system, while avoiding the translation of viral nucleoproteins, which is more conducive to the cell-specific translation of viral membrane proteins. Thus, the VRV system has better packaging efficiency. Furthermore, since the viral membrane proteins can bind to corresponding cell receptors, and the expression of cell receptors varies across different tissues, the VRV delivery systems constructed using different viral membrane proteins can achieve targeting of different tissues, thereby endowing the VRV system with tissue-targeted delivery functionality. Therefore, the VRV delivery system constructed in the present disclosure can be modified with different elements to develop a modular platform with high efficiency and low immunogenicity for different diseases and different targets. By efficiently delivering saRNA into cells or the body, the efficient and sustained expression of the carried target proteins is achieved.

### BRIEF DESCRIPTION OF THE DRAWINGS

FIGS. 1A-1B show a schematic diagram of the construction of a novel saRNA delivery (VRV) system mediated by viral envelope protein-assembled microvesicles and a schematic diagram of microvesicles of different viral envelopes obtained from this VRV system, wherein FIG. 1A: a schematic diagram of microvesicles formed after co-transfection of saRNA and viral envelope protein expression plasmids and their delivery into cells to express target proteins; FIG. 1B: microvesicles formed by different viral envelope proteins constructed by this VRV platform.

FIG. 2 shows the packaging efficiency and the expression of target proteins of different viral envelope proteins in the VRV system in naïve BHK-21 cells, including Chikungunya virus (CHIKV)-E, VEEV-E, Ebola virus (EBOV)-GP, Nipah virus (NIV)-GF, Lassa virus (LASV)-GP, Rabies virus (RABV)-G, H7N1-HANA, Respiratory syncytial virus (RSV)-F, where the expression of the target proteins is indicated by the fluorescent protein EGFP.

FIGS. 3A-3E show the electron microscopic morphology of microvesicles of different viral envelopes constructed by the VRV system, wherein FIG. 3A: a comparison of the electron microscopic structure of the VEEV virus TC-83 strain and the electron microscopic structure of VEEVrep-VEEV-E microvesicles assembled by SaRNA-VEEV envelope glycoprotein E; FIG. 3B: the electron microscopic structure of VEEVrep-RABV-G microvesicles assembled by VEEV replicon-rabies virus envelope glycoprotein G; FIG. 3C: the electron microscopic structure of VEEVrep-EBOV-GP microvesicles assembled by VEEV replicon-Ebola virus envelope glycoprotein G; FIG. 3D: the electron microscopic structure of VEEVrep-NIV-GF microvesicles assembled by VEEV replicon-Nipah virus envelope glycoprotein GF; FIG. 3E: the electron microscopic structure of VEEVrep-CHIKV-E microvesicles assembled by VEEV replicon-Chikungunya virus envelope glycoprotein E. Bar: 500 nm and 200 nm.

FIGS. 4A-4B show the negative staining electron microscopic structure and particle size of VEEVrep-VEEV-E microvesicle membranes obtained via the VRV system, wherein FIG. 4A: a comparison of the electron microscopic structure of the VEEV virus TC-83 strain and the negative staining electron microscopic structure of VEEVrep-VEEV-E microvesicles; FIG. 4B: the particle size of VEEVrep-VEEV-E microvesicles detected by DLS. Bar: 200 nm and 25 nm.

FIGS. 5A-5B show the strategy for delivering target proteins using the VRV system and the delivery of human angiotensin-converting enzyme II (hACE2) achieved by this strategy, wherein FIG. 5A: a schematic diagram of introducing target proteins after saRNA and the types of optional target proteins; FIG. 5B: a delivery of hACE2 loaded in saRNA via the VRV system and the verification of the hACE2 expression after transfection with microvesicles.

### DETAILED DESCRIPTION OF THE EMBODIMENTS

In the following examples of the present disclosure, the experimental methods without specifying specific conditions are generally carried out under conventional conditions or the conditions recommended by the manufacturers. Various commonly used chemical and biological reagents used in the examples are commercially available products.

Unless otherwise defined, all technical and scientific terms used in the present disclosure have the same meanings as commonly understood by those skilled in the technical field to which the present disclosure belongs. The terms used in the description of the present disclosure are only for the purpose of describing specific examples and are not intended to limit the present disclosure.

The terms “including” and “having” and any variations thereof used herein are intended to cover non-exclusive inclusions. For example, a process, method, device, product or equipment that includes a series of steps is not limited to the steps or modules listed, but optionally further includes unlisted steps, or optionally further includes other steps inherent to the process, method, product, or equipment.

The term “more” mentioned herein refers to two or more. “And/or” describes the association relationship of associated objects, indicating that there can be three types of relationships, for example, A and/or B can represent three scenarios: A exists alone, A and B exist simultaneously, and B exists alone. The character “/” generally refers to that the associated objects before and after are in an “or” relationship. Meanwhile, for a better understanding of the present disclosure, definitions and explanations of relevant terms are provided below.

The term “vector” used herein refers to a nucleic acid carrier tool into which polynucleotides can be inserted. When a vector enables the expression of a protein encoded by the inserted polynucleotide, the vector is called an expression vector. Vectors can be introduced into host cells by transformation, transduction, or transfection, enabling the genetic material elements they carry to be expressed in the host cells. Vectors are well-known to those skilled in the art, including but not limited to: plasmids; phagemids; cosmids; artificial chromosomes, such as yeast artificial chromosomes (YACs), bacterial artificial chromosomes (BACs) or P1-derived artificial chromosomes (PACs); phages such as X phages or M13 phages, and animal viruses, etc. Animal viruses that can be used as vectors include, but are not limited to, retroviruses (including lentiviruses), adenoviruses, adeno-associated viruses, herpes viruses (such as herpes simplex virus), poxviruses, baculoviruses, papillomaviruses, and papovaviruses (such as SV40). A vector may contain multiple elements that control expression, including but not limited to promoter sequences, transcription initiation sequences, enhancer sequences, selection elements and reporter genes. In addition, the vector may also contain an origin of replication.

The term “host cell” used herein refers to a recipient cell that accepts exogenous genes during transformation and transduction (infection), including but not limited to, prokaryotic cells such as Escherichia coli or Bacillus subtilis, fungal cells such as yeast cells or Aspergillus, insect cells such as Drosophila S2 cells or Sf9 cells, or animal cells such as fibroblasts, CHO cells, COS cells, NSO cells, HeLa cells, BHK cells, HEK 293 cells or human cells, etc.

The term “microvesicles” used herein refers to extracellular vehicles (EVs) formed by direct cell budding outward or “extrusion” from cells, containing cell membranes and partial cytoplasmic components. The particle size of EVs ranges from 30 nm-1000 nm, and the surface contains proteins such as CD40, integrins, selectins, etc. The microvesicles described herein are a type of EVs including viral envelope proteins, with an average particle size of about 58 nm.

The term “saRNA” used herein, i.e., self-amplifying RNA, is a short single-stranded oligonucleotide that can self-replicate using its own RNA sequence as a template. In addition, saRNA also carries a sequence capable of expressing RNA polymerase (RNA-dependent RNA polymerase, RdRP).

The term “core region” used herein refers to the capsid core region formed by viral capsid proteins (CAs). Taking mature and infectious HIV-1 as an example, the HIV-1 viral genome is located within a conical capsid core composed of viral capsid proteins. The capsid core is assembled from approximately 250 CA hexamers and 12 pentamers. The CA subunit is further composed of two structural domains, namely the N-terminal domain (NTD) and the C-terminal domain (CTD).

In one aspect, the present disclosure provides a use of alphavirus replicons and viral envelope proteins in constructing a saRNA delivery system. The saRNA delivery system is a microvesicle encapsulating saRNA, and the saRNA includes a target gene sequence. The microvesicle is a vesicular body with a bilayer membrane structure assembled from a viral envelope protein and a lipid bilayer, not including a capsid protein.

In one aspect, the present disclosure provides a method for constructing a saRNA microvesicle delivery system, including the following steps:
(1) designing a saRNA expression vector 1 and a membrane protein expression vector 2;
(2) co-transfecting the expression vector 1 and the expression vector 2 into a host cell;
(3) culturing the host cell and isolating to obtain a microvesicle containing saRNA;
wherein the saRNA includes a target gene sequence;
the replicon in the saRNA expression vector is an alphavirus replicon;
the microvesicle does not include a capsid protein.

In some embodiments, the nucleotide sequence of the alphavirus replicon is as shown in SEQ ID NO: 3.

In some embodiments, the expression vector includes plasmids, phages and viruses.

In some embodiments, the expression vector of the saRNA including the target gene sequence is a plasmid.

In some embodiments, the expression vector of the gene sequence of the viral envelope protein is a plasmid.

In some embodiments, the membrane protein is a viral membrane protein.

In some embodiments, the viral membrane protein is selected from the viruses of Alphavirus, Ebola virus, Arenavirus, Henipavirus, Lyssavirus, Pneumovirus, Alpharetrovirus, Lentivirus, Vesiculovirus, Marburgvirus, Mumpsvirus, Morbillivirus, Influenzavirus A, Influenzavirus B, Influenzavirus C, Hantavirus, Flavivirus, Hepacivirus, and/or Rubivirus.

In some embodiments, the viral membrane protein is selected from CHIKV, VEEV, EBOV, NIV, LASV, RABV, RSV and/or H7N1 viruses.

In some embodiments, the target gene is a therapeutic gene or an antigen gene.

In some embodiments, the therapeutic gene is an immunostimulatory gene or a suicide gene.

In some embodiments, the immunostimulatory gene encodes an immunostimulatory protein;

In some embodiments, the immunostimulatory protein is selected from cytokines, chemokines, interferons, tumor necrosis factors (TNF), colony-stimulating factors, proteins exposed to APCs (antigen-presenting cells), growth factors, Class I or Class II major histocompatibility complex (MHC) components, inducers or inhibitors of apoptosis, cell growth inhibitors, immunotoxins, and blockers of immune escape mechanisms of immune checkpoint inhibitors;

In some embodiments, the cytokine is an interleukin; more preferably, the cytokine is selected from IL-2, IL-6, IL-12, IL-15, and IL-24;

In some embodiments, the chemokine is selected from CXCL10, CXCL9, and CXCL11;

In some embodiments, the interferon is selected from IFN7 and IFNa;

In some embodiments, the colony-stimulating factor is selected from GM-CSF, G-CSF, and M-CSF;

In some embodiments, the protein of the APC (antigen-presenting cell) is selected from B7.1 and B7.2;

In some embodiments, the growth factor is selected from transforming growth factor TGF, fibroblast growth factor FGF, and vascular endothelial growth factor VEGF;

In some embodiments, the inducer or inhibitor of apoptosis is selected from one or more of Bax, Bcl2, and/or BclX;

In some embodiments, the cell growth inhibitor is selected from p21, p16, and Rb;

In some embodiments, the suicide gene is selected from thymidine kinase (TK), thymidylate kinase, cytosine deaminase (CDase), and uracil phosphoribosyltransferase (UPRTase);

In some embodiments, the antigen gene is a receptor gene, such as hACE2;

In some embodiments, the nucleotide sequence of hACE2 is as shown in SEQ ID NO: 15.

In another aspect, the present disclosure provides a microvesicle delivery vector prepared by the aforementioned method, wherein the delivery vector includes a microvesicle with a viral membrane protein embedded on the surface and saRNA packaged in the vesicle, the microvesicle does not include a capsid protein.

In some embodiments, the saRNA in the microvesicle delivery vector further includes a target gene sequence.

In some embodiments, the microvesicle is a vesicular body with a bilayer membrane structure assembled from a viral envelope protein and a lipid bilayer.

In some embodiments, the particle size of the microvesicle is about 10 nm-1000 nm.

In some embodiments, the particle size of the microvesicle is about 50-100 nm.

In some embodiments, the particle size of the microvesicle is about 58 nm.

In another aspect, the present disclosure provides a cell that stably expresses the aforementioned microvesicle delivery vector.

In another aspect, the present disclosure provides a use of the aforementioned method, microvesicle delivery vector, and/or cell in the manufacture of a targeted drug.

In another aspect, the present disclosure provides a use of the aforementioned method, microvesicle delivery vector, and/or cell in the manufacture of a vaccine preparation.

### EXAMPLES

The technical solutions of the present disclosure will be further described below through specific embodiments. Those skilled in the art should understand that the examples are only for helping understand the present disclosure and should not be regarded as specific limitations on the present disclosure.

The experimental methods involved in this section, such as PCR, enzyme digestion, ligation, transformation, RNA extraction, and RT-PCR, etc., unless otherwise specified, all adopt conventional methods in the field. The following enumerated are only several specific examples of the present disclosure. Obviously, the present disclosure is not limited to the following examples, and there can be many variations. Therefore, modifications or improvements made by those skilled in the art on the basis of the content of the present disclosure shall all fall within the scope of protection claimed by the present disclosure.

### Example 1: Construction of a Novel SaRNA Delivery System Mediated by Viral Envelope Protein-Assembled Microvesicles

During the exploration of the present disclosure, it was found that the RNA fragments of alphavirus replicons in host cells can be mediated to be packaged by the envelope proteins of various viruses to form cellular microvesicles. Therefore, the present disclosure utilizes the assembly ability of viral envelope proteins and the self-replication ability of the alphavirus genome, after modification and design, constructs a novel saRNA system delivery platform mediated by viral envelope protein-assembled microvesicles (Virus-Replicon vesicles, VRV) for packaging and delivering saRNA, and realizes the expression of downstream target proteins through saRNA.

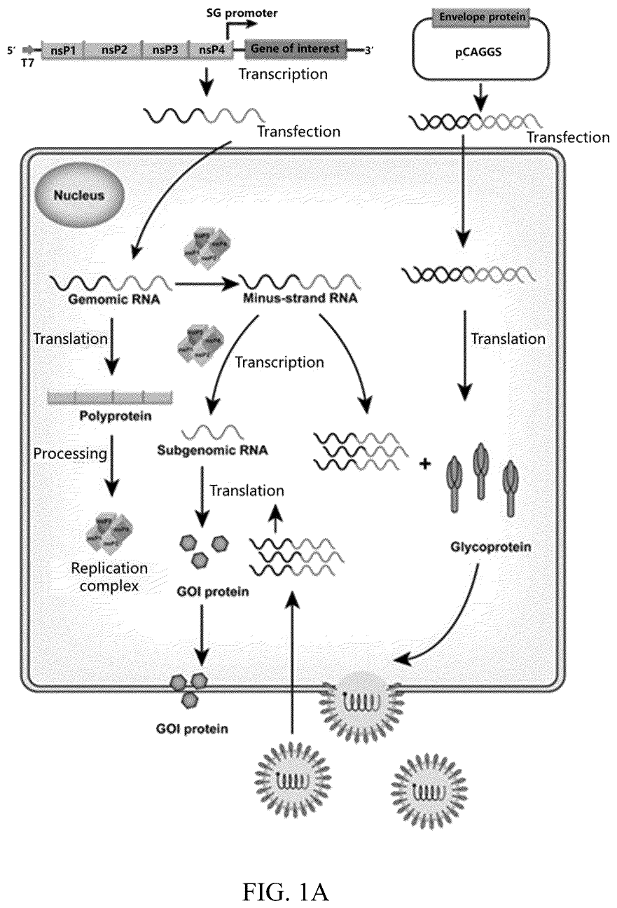

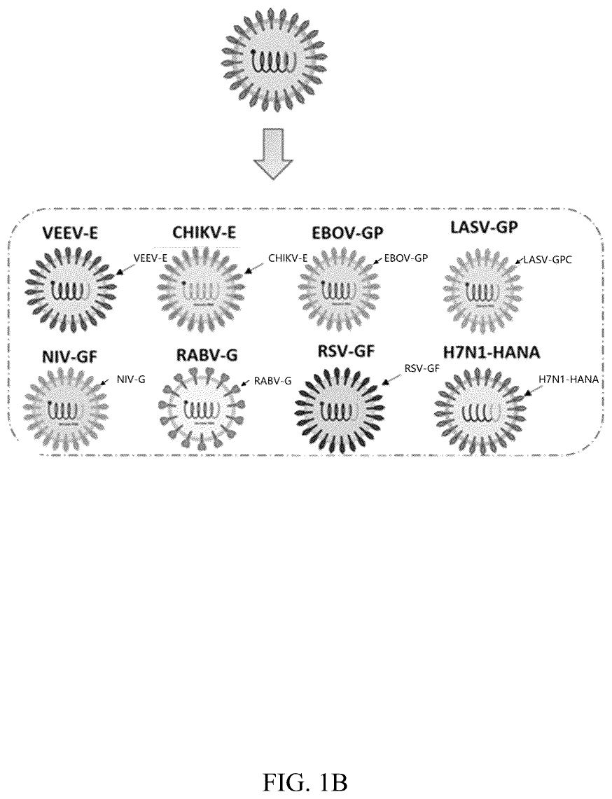

The specific construction schematic diagram is shown in FIGS. 1A-1B. The saRNA-GOI plasmid expressing the target protein (Gene of Interest, GOI) is constructed by reverse genetics technology, and saRNA-GOI RNA is obtained through in vitro transcription. The saRNA-GOI RNA and the eukaryotic expression plasmid expressing the viral envelope protein are co-transcribed in host cells. After entering the cell, the saRNA-GOI RNA is translated into a replication complex. Under the action of the replication complex, the progeny RNA and the target protein are expressed; at the same time, the envelope protein expression plasmid entering the host cell expresses the envelope proteins. The envelope proteins self-assemble near the lipid membrane to form microvesicles encapsulating saRNA. In the present disclosure, microvesicles assembled by different viral envelope proteins are constructed through this VRV system construction method, including CHIKV, VEEV, EBOV, NIV, LASV, RABV, H7N1, and RSV (see FIG. 1).

### Example 2: Packaging Efficiency and Expression of Target Proteins of Different Viral Envelope Proteins in the VRV System

According to the strategy in Example 1, the VEEV replicon RNA (nsp1-nsp4) expressing EGFP plasmid (pacyc-VEEVrep-EGFP) was firstly constructed in this Example, whose nucleotide sequence is shown in SEQ ID NO: 1, wherein the vector sequence is shown in SEQ ID NO: 2, the VEEVrep sequence is shown in SEQ ID NO: 3, and the EGFP sequence is shown in SEQ ID NO: 4. The construction method is as follows: using the full-length infectious clone of VEEV vaccine strain pacyc-TC-83 (preserved in our laboratory) as a template, the structural protein region sequence in TC-83 was replaced with the EGFP sequence by using AscI and PacI restriction enzyme sites to obtain the pacyc-VEEVrep-EGFP clone. The pacyc-VEEVrep-EGFP was linearized through the NotI restriction enzyme site. VEEVrep-EGFP saRNA was obtained by in vitro transcription of RNA using the T7 mMESSAGE mMACHINE kit (TAKARA).

The nucleotide sequence of SEQ ID NO: 1 is as follows:

ATGggcggcgcatgagagaagcccagaccaattacctacccaaaatggagaaagttcacgttgacatcgaggaagacag
cccattcctcagagctttgcagcggagcttcccgcagtttgaggtagaagccaagcaggtcactgataatgaccatgctaatgccagagcgt
tttcgcatctggcttcaaaactgatcgaaacggaggtggacccatccgacacgatccttgacattggaagtgcgcccgcccgcagaatgtat
tctaagcacaagtatcattgtatctgtccgatgagatgtgcggaagatccggacagattgtataagtatgcaactaagctgaagaaaaactgta
aggaaataactgataaggaattggacaagaaaatgaaggagctcgccgccgtcatgagcgaccctgacctggaaactgagactatgtgcc
tccacgacgacgagtcgtgtcgctacgaagggcaagtcgctgtttaccaggatgtatacgcggttgacggaccgacaagtctctatcacca
agccaataagggagttagagtcgcctactggataggctttgacaccaccccttttatgtttaagaacttggctggagcatatccatcatactcta
ccaactgggccgacgaaaccgtgttaacggctcgtaacataggcctatgcagctctgacgttatggagcggtcacgtagagggatgtccat
tcttagaaagaagtatttgaaaccatccaacaatgttctattctctgttggctcgaccatctaccacgagaagagggacttactgaggagctgg
cacctgccgtctgtatttcacttacgtggcaagcaaaattacacatgtcggtgtgagactatagttagttgcgacgggtacgtcgttaaaagaat
agctatcagtccaggcctgtatgggaagccttcaggctatgctgctacgatgcaccgcgagggattcttgtgctgcaaagtgacagacacat
tgaacggggagagggtctcttttcccgtgtgcacgtatgtgccagctacattgtgtgaccaaatgactggcatactggcaacagatgtcagtg
cggacgacgcgcaaaaactgctggttgggctcaaccagcgtatagtcgtcaacggtcgcacccagagaaacaccaataccatgaaaaatt
accttttgcccgtagtggcccaggcatttgctaggtgggcaaaggaatataaggaagatcaagaagatgaaaggccactaggactacgag
atagacagttagtcatggggtgttgttgggcttttagaaggcacaagataacatctatttataagcgcccggatacccaaaccatcatcaaagt
gaacagcgatttccactcattcgtgctgcccaggataggcagtaacacattggagatcgggctgagaacaagaatcaggaaaatgttagag
gagcacaaggagccgtcacctctcattaccgccgaggacgtacaagaagctaagtgcgcagccgatgaggctaaggaggtgcgtgaag
ccgaggagttgcgcgcagctctaccacctttggcagctgatgttgaggagcccactctggaagccgatgtcgacttgatgttacaagaggct
ggggccggctcagtggagacacctcgtggcttgataaaggttaccagctacgctggcgaggacaagatcggctcttacgctgtgctttctc
cgcaggctgtactcaagagtgaaaaattatcttgcatccaccctctcgctgaacaagtcatagtgataacacactctggccgaaaagggcgtt
atgccgtggaaccataccatggtaaagtagtggtgccagagggacatgcaatacccgtccaggactttcaagctctgagtgaaagtgccac
cattgtgtacaacgaacgtgagttcgtaaacaggtacctgcaccatattgccacacatggaggagcgctgaacactgatgaagaatattaca
aaactgtcaagcccagcgagcacgacggcgaatacctgtacgacatcgacaggaaacagtgcgtcaagaaagaactagtcactgggcta
gggctcacaggcgagctggtggatcctcccttccatgaattcgcctacgagagtctgagaacacgaccagccgctccttaccaagtaccaa
ccataggggtgtatggcgtgccaggatcaggcaagtctggcatcattaaaagcgcagtcaccaaaaaagatctagtggtgagcgccaaga
aagaaaactgtgcagaaattataagggacgtcaagaaaatgaaagggctggacgtcaatgccagaactgtggactcagtgctcttgaatgg
atgcaaacaccccgtagagaccctgtatattgacgaagcttttgcttgtcatgcaggtactctcagagcgctcatagccattataagacctaaa
aaggcagtgctctgcggggatcccaaacagtgcggtttttttaacatgatgtgcctgaaagtgcattttaaccacgagatttgcacacaagtctt
ccacaaaagcatctctcgccgttgcactaaatctgtgacttcggtcgtctcaaccttgttttacgacaaaaaaatgagaacgacgaatccgaaa
gagactaagattgtgattgacactaccggcagtaccaaacctaagcaggacgatctcattctcacttgtttcagagggtgggtgaagcagttg
caaatagattacaaaggcaacgaaataatgacggcagctgcctctcaagggctgacccgtaaaggtgtgtatgccgttcggtacaaggtga
atgaaaatcctctgtacgcacccacctcagaacatgtgaacgtcctactgacccgcacggaggaccgcatcgtgtggaaaacactagccg
gcgacccatggataaaaacactgactgccaagtaccctgggaatttcactgccacgatagaggagtggcaagcagagcatgatgccatca
tgaggcacatcttggagagaccggaccctaccgacgtcttccagaataaggcaaacgtgtgttgggccaaggctttagtgccggtgctgaa
gaccgctggcatagacatgaccactgaacaatggaacactgtggattattttgaaacggacaaagctcactcagcagagatagtattgaacc
aactatgcgtgaggttctttggactcgatctggactccggtctattttctgcacccactgttccgttatccattaggaataatcactgggataact
ccccgtcgcctaacatgtacgggctgaataaagaagtggtccgtcagctctctcgcaggtacccacaactgcctcgggcagttgccactgga
agagtctatgacatgaacactggtacactgcgcaattatgatccgcgcataaacctagtacctgtaaacagaagactgcctcatgctttagtcc
tccaccataatgaacacccacagagtgacttttcttcattcgtcagcaaattgaagggcagaactgtcctggtggtcggggaaaagttgtccg
tcccaggcaaaatggttgactggttgtcagaccggcctgaggctaccttcagagctcggctggatttaggcatcccaggtgatgtgcccaaa
tatgacataatatttgttaatgtgaggaccccatataaataccatcactatcagcagtgtgaagaccatgccattaagcttagcatgttgaccaa
gaaagcttgtctgcatctgaatcccggcggaacctgtgtcagcataggttatggttacgctgacagggccagcgaaagcatcattggtgcta
tagcgcggcagttcaagttttcccgggtatgcaaaccgaaatcctcacttgaagagacggaagttctgtttgtattcattgggtacgatcgcaa
ggcccgtacgcacaatccttacaagctttcatcaaccttgaccaacatttatacaggttccagactccacgaagccggatgtgcaccctcatat
catgtggtgcgaggggatattgccacggccaccgaaggagtgattataaatgctgctaacagcaaaggacaacctggcggaggggtgtg
cggagcgctgtataagaagttcccggaaagcttcgatttacagccgatcgaagtaggaaaagcgcgactggtcaaaggtgcagctaaaca
tatcattcatgccgtaggaccaaacttcaacaaagtttcggaggttgaaggtgacaaacagttggcagaggcttatgagtccatcgctaagat
tgtcaacgataacaattacaagtcagtagcgattccactgttgtccaccggcatcttttccgggaacaaagatcgactaacccaatcattgaac
catttgctgacagctttagacaccactgatgcagatgtagccatatactgcagggacaagaaatgggaaatgactctcaaggaagcagtgg
ctaggagagaagcagtggaggagatatgcatatccgacgactcttcagtgacagaacctgatgcagagctggtgagggtgcatccgaaga
gttctttggctggaaggaagggctacagcacaagcgatggcaaaactttctcatatttggaagggaccaagtttcaccaggggccaagga
tatagcagaaattaatgccatgtggcccgttgcaacggaggccaatgagcaggtatgcatgtatatcctcggagaaagcatgagcagtatta
ggtcgaaatgccccgtcgaagagtcggaagcctcctcaccacctagcacgctgccttgcttgtgcatccatgccatgactccagaaagagt
acagcgcctaaaagcctcacgtccagaacaaattactgtgtgctcatcctttccattgccgaagtatagaatcactggtgtgcagaagatcca
atgctcccagcctatattgttctcaccgaaagtgcctgcgtatattcatccaaggaagtatctcgtggaaacaccaccggtagacgagactcc
ggagccatcggcagagaaccaatccacagaggggacacctgaacaaccaccacttataaccgaggatgagaccaggactagaacgcct
gagccgatcatcatcgaagaggaagaagaggatagcataagtttgctgtcagatggcccgacccaccaggtgctgcaagtcgaggcaga
cattcacgggccgccctctgtatctagctcatcctggtccattcctcatgcatccgactttgatgtggacagtttatccatacttgacaccctgga
gggagctagcgtgaccagcggggcaacgtcagccgagactaactcttacttcgcaaagagtatggagtttctggcgcgaccggtgcctgc
gcctcgaacagtattcaggaaccctccacatcccgctccgcgcacaagaacaccgtcacttgcacccagcagggcctgctcgagaacca
gcctagtttccaccccgccaggcgtgaatagggtgatcactagagaggagctcgaggcgcttaccccgtcacgcactcctagcaggtcgg
tctcgagaaccagcctggtctccaacccgccaggcgtaaatagggtgattacaagagaggagtttgaggcgttcgtagcacaacaacaatg
acggtttgatgcgggtgcatacatcttttcctccgacaccggtcaagggcatttacaacaaaaatcagtaaggcaaacggtgctatccgaagt
ggtgttggagaggaccgaattggagatttcgtatgccccgcgcctcgaccaagaaaaagaagaattactacgcaagaaattacagttaaatc
ccacacctgctaacagaagcagataccagtccaggaaggtggagaacatgaaagccataacagctagacgtattctgcaaggcctaggg
cattatttgaaggcagaaggaaaagtggagtgctaccgaaccctgcatcctgttcctttgtattcatctagtgtgaaccgtgccttctcaagccc
caaggtcgcagtggaagcctgtaacgccatgttgaaagagaactttccgactgtggcttcttactgtattattccagagtacgatgcctatttgg
acatggttgacggagcttcatgctgcttagacactgccagtttttgccctgcaaagctgcgcagctttccaaagaaacactcctatttggaacc
cacaatacgatcggcagtgccttcagcgatccagaacacgctccagaacgtcctggcagctgccacaaaaagaaattgcaatgtcacgca
aatgagagaattgcccgtattggattcggcggcctttaatgtggaatgcttcaagaaatatgcgtgtaataatgaatattgggaaacgtttaaag
aaaaccccatcaggcttactgaagaaaacgtggtaaattacattaccaaattaaaaggaccaaaagctgctgctctttttgcgaagacacata
atttgaatatgttgcaggacataccaatggacaggtttgtaatggacttaaagagagacgtgaaagtgactccaggaacaaaacatactgaa
gaacggcccaaggtacaggtgatccaggctgccgatccgctagcaacagcgtatctgtgcggaatccaccgagagctggttaggagatta
aatgcggtcctgcttccgaacattcatacactgtttgatatgtcggctgaagactttgacgctattatagccgagcacttccagcctggggattg
tgttctggaaactgacatcgcgtcgtttgataaaagtgaggacgacgccatggctctgaccgcgttaatgattctggaagacttaggtgtgga
cgcagagctgttgacgctgattgaggcggctttcggcgaaatttcatcaatacatttgcccactaaaactaaatttaaattcggagccatgatg
aaatctggaatgttcctcacactgtttgtgaacacagtcattaacattgtaatcgcaagcagagtgttgagagaacggctaaccggatcaccat
gtgcagcattcattggagatgacaatatcgtgaaaggagtcaaatcggacaaattaatggcagacaggtgcgccacctggttgaatatggaa
gtcaagattatagatgctgtggtgggcgagaaagcgccctatttctgtggagggtttattttgtgtgactccgtgaccggcacagcgtgccgt
gtggcagaccccctaaaaaggctgtttaagcttggcaaacctctggcagcagacgatgaacatgatgatgacaggagaagggcattgcat
gaagagtcaacacgctggaaccgagtgggtattctttcagagctgtgcaaggcagtagaatcaaggtatgaaaccgtaggaacttccatcat
agttatggccatgactactctagctagcagtgttaaatcattcagctacctgagaggggcccctataactctctacggctaacctgaatggact
acgacatcgatggcgcgccACCATGGTGAGCAAGGGCGAGGAGCTGTTCACCGGGGTGGTGCC
CATCCTGGTCGAGCTGGACGGCGACGTAAACGGCCACAAGTTCAGCGTGTCCGGCG
AGGGCGAGGGCGATGCCACCTACGGCAAGCTGACCCTGAAGTTCATCTGCACCACCG
GCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAG
ACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACT
ACAACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTACC
AGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTG
CTGGAGTTCGTGACCGCCGCCGGGATCACTCTCGGCATGGACGAGCTGTACAAGGCT
AGCATCGAGGGCCGGGGCGGCTGGGGCAAAGAGACAGCCGCCGCCAAGTTCGAGC
GGCAGCACATGGACAGCTGATAAttaattaagtaaccgatacagcagcaattggcaagctgcttacatagaactcgcg
gcgattggcatgccgctttaaaatttttattttattttttcttttcttttccgaatcggattttgtttttaatatttcaaaaaaaaaaaaaaaaa
aaaaaaaaaaaaaaaaaaaaaaaaaaaaaaaaaggaagagcgcggccgcaTCCGGCGTTCAGCCTGTGCCACAGCCGA
CAGGATGGTGACCACCATTTGCCCCATATCACCGTCGGTACTGATCCCGTCGTCAATA
AACCGAACCGCTACACCCTGAGCATCAAACTCTTTTATCAGTTGGATCATGTCGGCGG
TGTCGCGGCCAAGACGGTCGAGCTTCTTCACCAGAATGACATCACCTTCCTCCACCTT
CATCCTCAGCAAATCCAGCCCTTCCCGATCTGTTGAACTGCCGGATGCCTTGTCGGTA
AAGATGCGGTTAGCTTTTACCCCTGCATCTTTGAGCGCTGAGGTCTGCCTCGTGAAGA
AGGTGTTGCTGACTCATACCAGGCCTGAATCGCCCCATCATCCAGCCAGAAAGTGAG
GGAGCCACGGTTGATGAGAGCTTTGTTGTAGGTGGACCAGTTGGTGATTTTGAACTTT
TGCTTTGCCACGGAACGGTCTGCGTTGTCGGGAAGATGCGTGATCTGATCCTTCAACT
CAGCAAAAGTTCGATTTATTCAACAAAGCCGCCGTCCCGTCAAGTCAGCGTAATGCT
CTGCCAGTGTTACAACCAATTAACCAATTCTGATTAGAAAAACTCATCGAGCATCAAA
TGAAACTGCAATTTATTCATATCAGGATTATCAATACCATATTTTTGAAAAAGCCGTTTC
TGTAATGAAGGAGAAAACTCACCGAGGCAGTTCCATAGGATGGCAAGATCCTGGTAT
CGGTCTGCGATTCCGACTCGTCCAACATCAATACAACCTATTAATTTCCCCTCGTCAAA
AATAAGGTTATCAAGTGAGAAATCACCATGAGTGACGACTGAATCCGGTGAGAATGG
CAAAAGCTTATGCATTTCTTTCCAGACTTGTTCAACAGGCCAGCCATTACGCTCGTCA
TCAAAATCACTCGCATCAACCAAACCGTTATTCATTCGTGATTGCGCCTGAGCGAGAC
GAAATACGCGATCGCTGTTAAAAGGACAATTACAAACAGGAATCGAATGCAACCGGC
GCAGGAACACTGCCAGCGCATCAACAATATTTTCACCTGAATCAGGATATTCTTCTAA
TACCTGGAATGCTGTTTTCCCGGGGATCGCAGTGGTGAGTAACCATGCATCATCAGGA
GTACGGATAAAATGCTTGATGGTCGGAAGAGGCATAAATTCCGTCAGCCAGTTTAGTC
TGACCATCTCATCTGTAACATCATTGGCAACGCTACCTTTGCCATGTTTCAGAAACAA
CTCTGGCGCATCGGGCTTCCCATACAATCGATAGATTGTCGCACCTGATTGCCCGACAT
TATCGCGAGCCCATTTATACCCATATAAATCAGCATCCATGTTGGAATTTAATCGCGGCC
TCGAGCAAGACGTTTCCCGTTGAATATGGCTCATAACACCCCTTGTATTACTGTTTATG
TAAGCAGACAGTTTTATTGTTCATGATGATATATTTTTATCTTGTGCAATGTAACATCAG
AGATTTTGAGACACAACGTGGCTTTGTTGAATAAATCGAACTTTTGCTGAGTTGAAGG
ATCAGATCACGCATCTTCCCGACAACGCAGACCGTTCCGTGGCAAAGCAAAAGTTCA
AAATCACCAACTGGTCCACCTACAACAAAGCTCTCATCAACCGTGGCTCCCTCACTTT
CTGGCTGGATGATGGGGCGATTCAGGCCTGGTATGAGTCAGCAACACCTTCTTCACG
AGGCAGACCTCAGCGCTAGCGGAGTGTATACTGGCTTACTATGTTGGCACTGATGAGG
GTGTCAGTGAAGTGCTTCATGTGGCAGGAGAAAAAAGGCTGCACCGGTGCGTCAGC
AGAATATGTGATACAGGATATATTCCGCTTCCTCGCTCACTGACTCGCTACGCTCGGTC
GTTCGACTGCGGCGAGCGGAAATGGCTTACGAACGGGGCGGAGATTTCCTGGAAGAT
GCCAGGAAGATACTTAACAGGGAAGTGAGAGGGCCGCGGCAAAGCCGTTTTTCCATA
GGCTCCGCCCCCCTGACAAGCATCACGAAATCTGACGCTCAAATCAGTGGTGGCGAA
ACCCGACAGGACTATAAAGATACCAGGCGTTTCCCCCTGGCGGCTCCCTCGTGCGCT
CTCCTGTTCCTGCCTTTCGGTTTACCGGTGTCATTCCGCTGTTATGGCCGCGTTTGTCT
CATTCCACGCCTGACACTCAGTTCCGGGTAGGCAGTTCGCTCCAAGCTGGACTGTATG
CACGAACCCCCCGTTCAGTCCGACCGCTGCGCCTTATCCGGTAACTATCGTCTTGAGT
CCAACCCGGAAAGACATGCAAAAGCACCACTGGCAGCAGCCACTGGTAATTGATTTA
GAGGAGTTAGTCTTGAAGTCATGCGCCGGTTAAGGCTAAACTGAAAGGACAAGTTTT
GGTGACTGCGCTCCTCCAAGCCAGTTACCTCGGTTCAAAGAGTTGGTAGCTCAGAGA
ACCTTCGAAAAACCGCCCTGCAAGGCGGTTTTTTCGTTTTCAGAGCAAGAGATTACG
CGCAGACCAAAACGATCTCAAGAAGATCATCTTATTAAGGGGTCTGACGCTCAGTGG
AACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATT
TCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCA
GATTTATCAGCAATAAACCAGCCAGCCGGAAGGGCCGAGCGCAGAAGTGGTCCTGCA
ACTTTATCCGCCTCCATCCAGTCTATTAATTGTTGCCGGGAAGCTAGAGTAAGTAGTTC
GCCAGTTAATAGTTTGCGCAACGTTGTTGCCATTGCTGCAGGCATCGTGGTGTCACGC
TCGTCGTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTTGTCAG
AAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATT
CTGAGAATAGTGTATGCGGCGACCGAGTTGCTCTTGCCCGGCGTCAACACGGGATAA
TACCGCGCCACATAGCAGAACTTTAAAAGTGCTCATCATTGGAAAACGTTCTTCGGG
GCGAAAACTCTCAAGGATCTTACCGCTGTTGAGATCCAGTTCGATGTAACCCACTCGT
GCACCCAACTGATCTTCAGCATCTTTTACTTTCACCAGCGTTTCTGGGTGAGCAAAAA
CAGGAAGGCAAAATGCCGCAAAAAAGGGAATAAGGGCGACACGGAAATGTTGAATA
CTCATACTCTTCCTTTTTCAATATTATTGAAGCATTTATCAGGGTTATTGTCTCATGAGC
GGATACATATTTGAATGTATTTAGAAAAATAAACAAATAGGGGTTCCGCGCACATTTCC
CCGAAAAGTGCCACCTGACGTCTAAGAAACCATTATTATCATGACATTAACCTATAAA
AATAGGCGTATCACGAGGCCCTTTCGTCTTCAAGAATTtctagaggatccctaatacgactcactatag

The nucleotide sequence of SEQ ID NO: 2 is as follows:

ttaattaagtaaccgatacagcagcaattggcaagctgcttacatagaactcgcggcgattggcatgccgctttaaaatttttatttt
attttttcttttcttttccgaatcggattttgtttttaatatttcaaaaaaaaaaaaaaaaaaaaaaaaaaaaaaaaaaaaaaaaaaaaaaaa
aaggaagagcgcggccgcaTCCGGCGTTCAGCCTGTGCCACAGCCGACAGGATGGTGACCACCATT
TGCCCCATATCACCGTCGGTACTGATCCCGTCGTCAATAAACCGAACCGCTACACCCT
GAGCATCAAACTCTTTTATCAGTTGGATCATGTCGGCGGTGTCGCGGCCAAGACGGTC
GAGCTTCTTCACCAGAATGACATCACCTTCCTCCACCTTCATCCTCAGCAAATCCAGC
CCTTCCCGATCTGTTGAACTGCCGGATGCCTTGTCGGTAAAGATGCGGTTAGCTTTTA
CCCCTGCATCTTTGAGCGCTGAGGTCTGCCTCGTGAAGAAGGTGTTGCTGACTCATAC
CAGGCCTGAATCGCCCCATCATCCAGCCAGAAAGTGAGGGAGCCACGGTTGATGAGA
GCTTTGTTGTAGGTGGACCAGTTGGTGATTTTGAACTTTTGCTTTGCCACGGAACGGT
CTGCGTTGTCGGGAAGATGCGTGATCTGATCCTTCAACTCAGCAAAAGTTCGATTTAT
TCAACAAAGCCGCCGTCCCGTCAAGTCAGCGTAATGCTCTGCCAGTGTTACAACCAA
TTAACCAATTCTGATTAGAAAAACTCATCGAGCATCAAATGAAACTGCAATTTATTCAT
ATCAGGATTATCAATACCATATTTTTGAAAAAGCCGTTTCTGTAATGAAGGAGAAAAC
TCACCGAGGCAGTTCCATAGGATGGCAAGATCCTGGTATCGGTCTGCGATTCCGACTC
GTCCAACATCAATACAACCTATTAATTTCCCCTCGTCAAAAATAAGGTTATCAAGTGA
GAAATCACCATGAGTGACGACTGAATCCGGTGAGAATGGCAAAAGCTTATGCATTTC
TTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGT
TAAAAGGACAATTACAAACAGGAATCGAATGCAACCGGCGCAGGAACACTGCCAGC
GCATCAACAATATTTTCACCTGAATCAGGATATTCTTCTAATACCTGGAATGCTGTTTTC
CCGGGGATCGCAGTGGTGAGTAACCATGCATCATCAGGAGTACGGATAAAATGCTTG
ATGGTCGGAAGAGGCATAAATTCCGTCAGCCAGTTTAGTCTGACCATCTCATCTGTAA
CATCATTGGCAACGCTACCTTTGCCATGTTTCAGAAACAACTCTGGCGCATCGGGCTT
CCCATACAATCGATAGATTGTCGCACCTGATTGCCCGACATTATCGCGAGCCCATTTAT
ACCCATATAAATCAGCATCCATGTTGGAATTTAATCGCGGCCTCGAGCAAGACGTTTC
CCGTTGAATATGGCTCATAACACCCCTTGTATTACTGTTTATGTAAGCAGACAGTTTTA
TTGTTCATGATGATATATTTTTATCTTGTGCAATGTAACATCAGAGATTTTGAGACACAA
CGTGGCTTTGTTGAATAAATCGAACTTTTGCTGAGTTGAAGGATCAGATCACGCATCT
TCCCGACAACGCAGACCGTTCCGTGGCAAAGCAAAAGTTCAAAATCACCAACTGGT
CCACCTACAACAAAGCTCTCATCAACCGTGGCTCCCTCACTTTCTGGCTGGATGATGG
GGCGATTCAGGCCTGGTATGAGTCAGCAACACCTTCTTCACGAGGCAGACCTCAGCG
CTAGCGGAGTGTATACTGGCTTACTATGTTGGCACTGATGAGGGTGTCAGTGAAGTGC
TTCATGTGGCAGGAGAAAAAAGGCTGCACCGGTGCGTCAGCAGAATATGTGATACAG
GATATATTCCGCTTCCTCGCTCACTGACTCGCTACGCTCGGTCGTTCGACTGCGGCGA
GCGGAAATGGCTTACGAACGGGGCGGAGATTTCCTGGAAGATGCCAGGAAGATACTT
AACAGGGAAGTGAGAGGGCCGCGGCAAAGCCGTTTTTCCATAGGCTCCGCCCCCCT
GACAAGCATCACGAAATCTGACGCTCAAATCAGTGGTGGCGAAACCCGACAGGACT
ATAAAGATACCAGGCGTTTCCCCCTGGCGGCTCCCTCGTGCGCTCTCCTGTTCCTGCC
TTTCGGTTTACCGGTGTCATTCCGCTGTTATGGCCGCGTTTGTCTCATTCCACGCCTGA
CACTCAGTTCCGGGTAGGCAGTTCGCTCCAAGCTGGACTGTATGCACGAACCCCCCG
TTCAGTCCGACCGCTGCGCCTTATCCGGTAACTATCGTCTTGAGTCCAACCCGGAAAG
ACATGCAAAAGCACCACTGGCAGCAGCCACTGGTAATTGATTTAGAGGAGTTAGTCT
TGAAGTCATGCGCCGGTTAAGGCTAAACTGAAAGGACAAGTTTTGGTGACTGCGCTC
CTCCAAGCCAGTTACCTCGGTTCAAAGAGTTGGTAGCTCAGAGAACCTTCGAAAAAC
CGCCCTGCAAGGCGGTTTTTTCGTTTTCAGAGCAAGAGATTACGCGCAGACCAAAAC
GATCTCAAGAAGATCATCTTATTAAGGGGTCTGACGCTCAGTGGAACGAAAACTCAC
GTTAAGGGATTTTGGTCATGAGATTATCAAAAAGGATCTTCACCTAGATCCTTTTAAAT
TAAAAATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCATAG
TTGCCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCAATA
AACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGTT
TGCGCAACGTTGTTGCCATTGCTGCAGGCATCGTGGTGTCACGCTCGTCGTTTGGTAT
GGCTTCATTCAGCTCCGGTTCCCAACGATCAAGGCGAGTTACATGATCCCCCATGTTG
TGCAAAAAAGCGGTTAGCTCCTTCGGTCCTCCGATCGTTGTCAGAAGTAAGTTGGCC
GCAGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTGT
ATGCGGCGACCGAGTTGCTCTTGCCCGGCGTCAACACGGGATAATACCGCGCCACAT
AGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGAT
CTTCAGCATCTTTTACTTTCACCAGCGTTTCTGGGTGAGCAAAAACAGGAAGGCAAA
ATGCCGCAAAAAAGGGAATAAGGGCGACACGGAAATGTTGAATACTCATACTCTTCC
TTTTTCAATATTATTGAAGCATTTATCAGGGTTATTGTCTCATGAGCGGATACATATTTG
AATGTATTTAGAAAAATAAACAAATAGGGGTTCCGCGCACATTTCCCCGAAAAGTGCC
ACCTGACGTCTAAGAAACCATTATTATCATGACATTAACCTATAAAAATAGGCGTATCA
CGAGGCCCTTTCGTCTTCAAGAATTtctagaggatccctaatacgactcactatag

The nucleotide sequence of SEQ ID NO: 3 is as follows:

ATGggcggcgcatgagagaagcccagaccaattacctacccaaaatggagaaagttcacgttgacatcgaggaagacag
cccattcctcagagctttgcagcggagcttcccgcagtttgaggtagaagccaagcaggtcactgataatgaccatgctaatgccagagcgt
tttcgcatctggcttcaaaactgatcgaaacggaggtggacccatccgacacgatccttgacattggaagtgcgcccgcccgcagaatgtat
tctaagcacaagtatcattgtatctgtccgatgagatgtgcggaagatccggacagattgtataagtatgcaactaagctgaagaaaaactgta
aggaaataactgataaggaattggacaagaaaatgaaggagctcgccgccgtcatgagcgaccctgacctggaaactgagactatgtgcc
tccacgacgacgagtcgtgtcgctacgaagggcaagtcgctgtttaccaggatgtatacgcggttgacggaccgacaagtctctatcacca
agccaataagggagttagagtcgcctactggataggctttgacaccaccccttttatgtttaagaacttggctggagcatatccatcatactcta
ccaactgggccgacgaaaccgtgttaacggctcgtaacataggcctatgcagctctgacgttatggagcggtcacgtagagggatgtccat
tcttagaaagaagtatttgaaaccatccaacaatgttctattctctgttggctcgaccatctaccacgagaagagggacttactgaggagctgg
cacctgccgtctgtatttcacttacgtggcaagcaaaattacacatgtcggtgtgagactatagttagttgcgacgggtacgtcgttaaaagaat
agctatcagtccaggcctgtatgggaagccttcaggctatgctgctacgatgcaccgcgagggattcttgtgctgcaaagtgacagacacat
tgaacggggagagggtctcttttcccgtgtgcacgtatgtgccagctacattgtgtgaccaaatgactggcatactggcaacagatgtcagtg
cggacgacgcgcaaaaactgctggttgggctcaaccagcgtatagtcgtcaacggtcgcacccagagaaacaccaataccatgaaaaatt
accttttgcccgtagtggcccaggcatttgctaggtgggcaaaggaatataaggaagatcaagaagatgaaaggccactaggactacgag
atagacagttagtcatggggtgttgttgggcttttagaaggcacaagataacatctatttataagcgcccggatacccaaaccatcatcaaagt
gaacagcgatttccactcattcgtgctgcccaggataggcagtaacacattggagatcgggctgagaacaagaatcaggaaaatgttagag
gagcacaaggagccgtcacctctcattaccgccgaggacgtacaagaagctaagtgcgcagccgatgaggctaaggaggtgcgtgaag
ccgaggagttgcgcgcagctctaccacctttggcagctgatgttgaggagcccactctggaagccgatgtcgacttgatgttacaagaggct
ggggccggctcagtggagacacctcgtggcttgataaaggttaccagctacgctggcgaggacaagatcggctcttacgctgtgctttctc
cgcaggctgtactcaagagtgaaaaattatcttgcatccaccctctcgctgaacaagtcatagtgataacacactctggccgaaaagggcgtt
atgccgtggaaccataccatggtaaagtagtggtgccagagggacatgcaatacccgtccaggactttcaagctctgagtgaaagtgccac
cattgtgtacaacgaacgtgagttcgtaaacaggtacctgcaccatattgccacacatggaggagcgctgaacactgatgaagaatattaca
aaactgtcaagcccagcgagcacgacggcgaatacctgtacgacatcgacaggaaacagtgcgtcaagaaagaactagtcactgggcta
gggctcacaggcgagctggtggatcctcccttccatgaattcgcctacgagagtctgagaacacgaccagccgctccttaccaagtaccaa
ccataggggtgtatggcgtgccaggatcaggcaagtctggcatcattaaaagcgcagtcaccaaaaaagatctagtggtgagcgccaaga
aagaaaactgtgcagaaattataagggacgtcaagaaaatgaaagggctggacgtcaatgccagaactgtggactcagtgctcttgaatgg
atgcaaacaccccgtagagaccctgtatattgacgaagcttttgcttgtcatgcaggtactctcagagcgctcatagccattataagacctaaa
aaggcagtgctctgcggggatcccaaacagtgcggtttttttaacatgatgtgcctgaaagtgcattttaaccacgagatttgcacacaagtctt
ccacaaaagcatctctcgccgttgcactaaatctgtgacttcggtcgtctcaaccttgttttacgacaaaaaaatgagaacgacgaatccgaaa
gagactaagattgtgattgacactaccggcagtaccaaacctaagcaggacgatctcattctcacttgtttcagagggtgggtgaagcagttg
caaatagattacaaaggcaacgaaataatgacggcagctgcctctcaagggctgacccgtaaaggtgtgtatgccgttcggtacaaggtga
atgaaaatcctctgtacgcacccacctcagaacatgtgaacgtcctactgacccgcacggaggaccgcatcgtgtggaaaacactagccg
gcgacccatggataaaaacactgactgccaagtaccctgggaatttcactgccacgatagaggagtggcaagcagagcatgatgccatca
tgaggcacatcttggagagaccggaccctaccgacgtcttccagaataaggcaaacgtgtgttgggccaaggctttagtgccggtgctgaa
gaccgctggcatagacatgaccactgaacaatggaacactgtggattattttgaaacggacaaagctcactcagcagagatagtattgaacc
aactatgcgtgaggttctttggactcgatctggactccggtctattttctgcacccactgttccgttatccattaggaataatcactgggataac
tccccgtcgcctaacatgtacgggctgaataaagaagtggtccgtcagctctctcgcaggtacccacaactgcctcgggcagttgccactgga
agagtctatgacatgaacactggtacactgcgcaattatgatccgcgcataaacctagtacctgtaaacagaagactgcctcatgctttagtcc
tccaccataatgaacacccacagagtgacttttcttcattcgtcagcaaattgaagggcagaactgtcctggtggtcggggaaaagttgtccg
tcccaggcaaaatggttgactggttgtcagaccggcctgaggctaccttcagagctcggctggatttaggcatcccaggtgatgtgcccaaa
tatgacataatatttgttaatgtgaggaccccatataaataccatcactatcagcagtgtgaagaccatgccattaagcttagcatgttgaccaa
gaaagcttgtctgcatctgaatcccggcggaacctgtgtcagcataggttatggttacgctgacagggccagcgaaagcatcattggtgcta
tagcgcggcagttcaagttttcccgggtatgcaaaccgaaatcctcacttgaagagacggaagttctgtttgtattcattgggtacgatcgcaa
ggcccgtacgcacaatccttacaagctttcatcaaccttgaccaacatttatacaggttccagactccacgaagccggatgtgcaccctcatat
catgtggtgcgaggggatattgccacggccaccgaaggagtgattataaatgctgctaacagcaaaggacaacctggcggaggggtgtg
cggagcgctgtataagaagttcccggaaagcttcgatttacagccgatcgaagtaggaaaagcgcgactggtcaaaggtgcagctaaaca
tatcattcatgccgtaggaccaaacttcaacaaagtttcggaggttgaaggtgacaaacagttggcagaggcttatgagtccatcgctaagat
tgtcaacgataacaattacaagtcagtagcgattccactgttgtccaccggcatcttttccgggaacaaagatcgactaacccaatcattgaac
catttgctgacagctttagacaccactgatgcagatgtagccatatactgcagggacaagaaatgggaaatgactctcaaggaagcagtgg
ctaggagagaagcagtggaggagatatgcatatccgacgactcttcagtgacagaacctgatgcagagctggtgagggtgcatccgaaga
gttctttggctggaaggaagggctacagcacaagcgatggcaaaactttctcatatttggaagggaccaagtttcaccaggcggccaagga
tatagcagaaattaatgccatgtggcccgttgcaacggaggccaatgagcaggtatgcatgtatatcctcggagaaagcatgagcagtatta
ggtcgaaatgccccgtcgaagagtcggaagcctcctcaccacctagcacgctgccttgcttgtgcatccatgccatgactccagaaagagt
acagcgcctaaaagcctcacgtccagaacaaattactgtgtgctcatcctttccattgccgaagtatagaatcactggtgtgcagaagatcca
atgctcccagcctatattgttctcaccgaaagtgcctgcgtatattcatccaaggaagtatctcgtggaaacaccaccggtagacgagactcc
ggagccatcggcagagaaccaatccacagaggggacacctgaacaaccaccacttataaccgaggatgagaccaggactagaacgcct
gagccgatcatcatcgaagaggaagaagaggatagcataagtttgctgtcagatggcccgacccaccaggtgctgcaagtcgaggcaga
cattcacgggccgccctctgtatctagctcatcctggtccattcctcatgcatccgactttgatgtggacagtttatccatacttgacaccctgg
agggagctagcgtgaccagcggggcaacgtcagccgagactaactcttacttcgcaaagagtatggagtttctggcgcgaccggtgcctgc
gcctcgaacagtattcaggaaccctccacatcccgctccgcgcacaagaacaccgtcacttgcacccagcagggcctgctcgagaacca
gcctagtttccaccccgccaggcgtgaatagggtgatcactagagaggagctcgaggcgcttaccccgtcacgcactcctagcaggtcgg
tctcgagaaccagcctggtctccaacccgccaggcgtaaatagggtgattacaagagaggagtttgaggcgttcgtagcacaacaacaatg
acggtttgatgcgggtgcatacatcttttcctccgacaccggtcaagggcatttacaacaaaaatcagtaaggcaaacggtgctatccgaagt
ggtgttggagaggaccgaattggagatttcgtatgccccgcgcctcgaccaagaaaaagaagaattactacgcaagaaattacagttaaatc
ccacacctgctaacagaagcagataccagtccaggaaggtggagaacatgaaagccataacagctagacgtattctgcaaggcctaggg
cattatttgaaggcagaaggaaaagtggagtgctaccgaaccctgcatcctgttcctttgtattcatctagtgtgaaccgtgccttctcaagccc
caaggtcgcagtggaagcctgtaacgccatgttgaaagagaactttccgactgtggcttcttactgtattattccagagtacgatgcctatttgg
acatggttgacggagcttcatgctgcttagacactgccagtttttgccctgcaaagctgcgcagctttccaaagaaacactcctatttggaacc
cacaatacgatcggcagtgccttcagcgatccagaacacgctccagaacgtcctggcagctgccacaaaaagaaattgcaatgtcacgca
aatgagagaattgcccgtattggattcggcggcctttaatgtggaatgcttcaagaaatatgcgtgtaataatgaatattgggaaacgtttaaag
aaaaccccatcaggcttactgaagaaaacgtggtaaattacattaccaaattaaaaggaccaaaagctgctgctctttttgcgaagacacata
atttgaatatgttgcaggacataccaatggacaggtttgtaatggacttaaagagagacgtgaaagtgactccaggaacaaaacatactgaa
gaacggcccaaggtacaggtgatccaggctgccgatccgctagcaacagcgtatctgtgcggaatccaccgagagctggttaggagatta
aatgcggtcctgcttccgaacattcatacactgtttgatatgtcggctgaagactttgacgctattatagccgagcacttccagcctggggattg
tgttctggaaactgacatcgcgtcgtttgataaaagtgaggacgacgccatggctctgaccgcgttaatgattctggaagacttaggtgtgga
cgcagagctgttgacgctgattgaggcggctttcggcgaaatttcatcaatacatttgcccactaaaactaaatttaaattcggagccatgatg
aaatctggaatgttcctcacactgtttgtgaacacagtcattaacattgtaatcgcaagcagagtgttgagagaacggctaaccggatcaccat
gtgcagcattcattggagatgacaatatcgtgaaaggagtcaaatcggacaaattaatggcagacaggtgcgccacctggttgaatatggaa
gtcaagattatagatgctgtggtgggcgagaaagcgccctatttctgtggagggtttattttgtgtgactccgtgaccggcacagcgtgccgt
gtggcagaccccctaaaaaggctgtttaagcttggcaaacctctggcagcagacgatgaacatgatgatgacaggagaagggcattgcat
gaagagtcaacacgctggaaccgagtgggtattctttcagagctgtgcaaggcagtagaatcaaggtatgaaaccgtaggaacttccatcat
agttatggccatgactactctagctagcagtgttaaatcattcagctacctgagaggggcccctataactctctacggctaacctgaatggact
acgacatcgatggcgcgccACC

The nucleotide sequence of SEQ ID NO: 4 is as follows:

ATGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGGCGAGGG
CGATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCCG
CTACCCCGACCACATGAAGCAGCACGACTTCTTCAAGTCCGCCATGCCCGAAGGCTA
CGTCCAGGAGCGCACCATCTTCTTCAAGGACGACGGCAACTACAAGACCCGCGCCG
AGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACTACAACAGCCA
CAACGTCTATATCATGGCCGACAAGCAGAAGAACGGCATCAAGGTGAACTTCAAGAT
CCGCCACAACATCGAGGACGGCAGCGTGCAGCTCGCCGACCACTACCAGCAGAACA
CCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTC
GTGACCGCCGCCGGGATCACTCTCGGCATGGACGAGCTGTACAAG

The nucleotide sequence of SEQ ID NO: 5 is as follows:

GTCGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGC
TGACCGCCCAACGACCCCCGCCCATTGACGTCAATAATGACGTATGTTCCCATAGTAA
CGCCAATAGGGACTTTCCATTGACGTCAATGGGTGGACTATTTACGGTAAACTGCCCA
CTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTG
GCAGTACATCTACGTATTAGTCATCGCTATTACCATGGGTCGAGGTGAGCCCCACGTTC
TGCTTCACTCTCCCCATCTCCCCCCCCTCCCCACCCCCAATTTTGTATTTATTTATTTTT
TAATTATTTTGTGCAGCGATGGGGGCGGGGGGGGGGGGGGCGCGCGCCAGGCGGGG
CGGGGCGGGGCGAGGGGCGGGGCGGGGCGAGGCGGAGAGGTGCGGCGGCAGCCAA
TCAGAGCGGCGCGCTCCGAAAGTTTCCTTTTATGGCGAGGCGGCGGCGGCGGCGGCC
CTATAAAAAGCGAAGCGCGCGGCGGGCGGGAGTCGCTGCGTTGCCTTCGCCCCGTGC
CCCGCTCCGCGCCGCCTCGCGCCGCCCGCCCCGGCTCTGACTGACCGCGTTACTCCC
ACAGGTGAGCGGGCGGGACGGCCCTTCTCCTCCGGGCTGTAATTAGCGCTTGGTTTA
ATGACGGCTCGTTTCTTTTCTGTGGCTGCGTGAAAGCCTTAAAGGGCTCCGGGAGGG
CCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCG
GGGCTTTGTGCGCTCCGCGTGTGCGCGAGGGGAGCGCGGCCGGGGGCGGTGCCCCG
CGGTGCGGGGGGGCTGCGAGGGGAACAAAGGCTGCGTGCGGGGTGTGTGCGTGGG
GGGGTGAGCAGGGGGTGTGGGCGCGGCGGTCGGGCTGTAACCCCCCCCTGCACCCC
CCTCCCCGAGTTGCTGAGCACGGCCCGGCTTCGGGTGCGGGGCTCCGTGCGGGGCGT
GGCGCGGGGCTCGCCGTGCCGGGCGGGGGGTGGCGGCAGGTGGGGGTGCCGGGCG
GGGCGGGGCCGCCTCGGGCCGGGGAGGGCTCGGGGGAGGGGCGCGGCGGCCCCGG
AGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGG
AGGCGCCGCCGCACCCCCTCTAGCGGGCGCGGGGCGAAGCGGTGCGGCGCCGGCAG
GAAGGAAATGGGCGGGGAGGGCCTTCGTGCGTCGCCGCGCCGCCGTCCCCTTCTCCC
TCTCCAGCCTCGGGGCTGTCCGCGGGGGGACGGCTGCCTTCGGGGGGGACGGGGCA
GGGCGGGGTTCGGCTTCTGGCGTGTGACCGGCGGCTCTAGAGCCTCTGCTAACCATG
TTCATGCCTTCTTCTTTTTCCTACAGCTCCTGGGCAACGTGCTGGTTATTGTGCTGTCT
CATCATTTTGGCAAAGAATTCGGCGGCCGCATGAGTCTGGCCATTCCAGTTATGTGCC
TGCTGGCAAATACCACGTTCCCCTGCTCCCAGCCCCCTTGCACACCCTGCTGCTACGA
AAAAGAGCCGGAGAAAACCTTGCGCATGCTTGAAGACAATGTCATGAGCCCCGGGT
ACTATCAGCTGCTACAAGCATCCTTAACATGTTCTCCCCGACGCCAGCGGCGCAGTAT
TAAGGACCACTTCAATGTCTATAAAGCCACAAGACCGTACCTAGCTCACTGTCCCGAC
TGTGGAGAAGGGCACTCGTGCCATAGTCCCGTAGCGCTAGAACGCATCAGAAACGAA
GCGACAGACGGGACGTTGAAAATCCAGGTTTCCTTGCAAATCGGAATAAAGACGGAT
GATAGCCATGATTGGACCAAGCTGCGTTATATGGACAATCACATGCCAGCAGACGCAG
AGCGGGCCGGGCTATTTGTAAGAACGTCAGCACCGTGCACGATTACTGGAACAATGG
GACACTTCATTCTGGCCCGATGTCCGAAAGGAGAAACTCTGACGGTAGGGTTCACTG
ACGGTAGGAAGATCAGTCACTCATGTACGCACCCATTTCACCATGACCCTCCTGTGAT
AGGCCGGGAAAAATTCCATTCCCGACCGCAGCACGGTAGGGAACTACCTTGCAGCAC
GTACGCGCAGAGCACCGCTGCAACTGCCGAGGAGATAGAGGTACATATGCCCCCAGA
CACCCCAGATCGCACATTAATGTCACAACAGTCCGGCAATGTAAAGATCACAGTCAAT
AGTCAGACGGTGCGGTACAAGTGCAATTGTGGTGACTCAAGTGAAGGATTAACCACT
ACAGATAAAGTGATTAATAACTGCAAGGTTGATCAATGCCATGCCGCGGTCACCAATC
ACAAAAAATGGCAGTATAATTCCCCTCTGGTCCCGCGTAATGCTGAATTCGGGGACCG
GAAAGGAAAAGTTCACATTCCATTTCCTCTGGCAAATGTGACATGCAGGGTGCCTAA
AGCAAGAAACCCCACCGTGACGTACGGAAAAAACCAAGTCATCATGTTGCTGTATCC
TGACCACCCAACGCTCCTGTCCTACAGGAATATGGGAGAAGAACCAAACTATCAAGA
AGAGTGGGTGACGCATAAGAAGGAGATCAGGTTAACCGTGCCGACTGAAGGGCTCG
AGGTCACGTGGGGTAACAATGAGCCGTACAAGTATTGGCCGCAGTTATCCACAAACG
GTACAGCCCACGGCCACCCGCATGAGATAATTCTGTATTATTATGAGCTGTACCCAACT
ATGACTGTGGTAGTTTTGTCAGTGGCCTCGTTCATACTCCTGTCGATGGTGGGTGTGG
CAGTGGGGATGTGCATGTGTGCACGACGCAGATGCATTACACCGTACGAACTGACAC
CAGGAGCTACCGTCCCTTTCCTGCTTAGCCTAATATGCTGCATTAGAACAGCTAAAGC
GGCCACATACCAGGAGGCCGCGGTATACCTGTGGAACGAGCAGCAGCCTTTATTTTG
GCTGCAAGCCCTTATTCCGCTGGCAGCCCTGATTGTCCTATGTAACTGTCTGAGACTC
TTACCATGCTGTTGTAAAATGTTGACTTTTTTAGCCGTACTGAGCGTCGGTGCCCACA
CTGTGAGTGCGTACGAACACGTAACAGTGATCCCGAACACGGTGGGAGTACCGTATA
AGACTCTAGTCAACAGACCGGGCTACAGCCCCATGGTATTGGAGATGGAGCTTCTGT
CTGTCACCTTGGAACCAACGCTATCGCTTGATTACATCACGTGCGAGTATAAAACCGT
TATCCCGTCTCCGTACGTGAAATGCTGCGGTACAGCAGAGTGTAAGGACAAGAGCCT
ACCTGATTACAGCTGTAAGGTCTTCACCGGCGTCTACCCATTCATGTGGGGCGGCGCC
TACTGCTTCTGCGACACCGAAAATACGCAATTGAGCGAAGCACATGTGGAGAAGTCC
GAATCATGCAAAACAGAATTTGCATCAGCATACAGGGCTCATACCGCATCCGCGTCAG
CTAAGCTCCGCGTCCTTTACCAAGGAAATAATATCACTGTAGCTGCTTATGCAAACGG
CGACCATGCCGTCACAGTTAAGGACGCTAAATTCATAGTGGGGCCAATGTCTTCAGCC
TGGACACCTTTCGACAATAAAATCGTGGTGTACAAAGGCGACGTCTACAACATGGAC
TACCCGCCCTTCGGCGCAGGAAGACCAGGACAATTTGGCGACATCCAAAGTCGCACG
CCTGAGAGCGAAGACGTCTATGCTAATACACAACTGGTACTGCAGAGACCGTCCGCG
GGTACGGTGCACGTGCCGTACTCTCAGGCACCATCTGGCTTCAAGTATTGGCTAAAAG
AACGAGGGGCGTCGCTGCAGCACACAGCACCATTTGGCTGTCAAATAGCAACAAAC
CCGGTAAGAGCGATGAACTGCGCCGTAGGGAACATGCCTATCTCCATCGACATACCGG
ACGCGGCCTTTACCAGGGTCGTCGACGCGCCATCTTTAACGGACATGTCGTGTGAGG
TATCAGCCTGCACCCATTCCTCAGACTTTGGGGGCGTAGCCATCATTAAATATGCAGCC
AGTAAGAAAGGCAAGTGTGCAGTGCACTCGATGACTAACGCCGTCACTATTCGGGAA
GCTGAAATAGAAGTAGAAGGGAACTCTCAGTTGCAAATCTCTTTTTCGACGGCCCTA
GCCAGCGCCGAATTTCGCGTACAAGTCTGTTCTACACAAGTACACTGTGCAGCCGAG
TGCCATCCACCGAAAGACCATATAGTCAATTACCCGGCGTCACACACCACCCTCGGG
GTCCAAGACATTTCCGCTACGGCGATGTCATGGGTGCAGAAGATCACGGGAGGTGTG
GGACTGGTTGTCGCTGTTGCAGCACTGATCCTAATCGTGGTGCTATGCGTGTCGTTTA
GCAGGCACTAAGCTAGCAGATCTTTTTCCCTCTGCCAAAAATTATGGGGACATCATGA
AGCCCCTTGAGCATCTGACTTCTGGCTAATAAAGGAAATTTATTTTCATTGCAATAGTG
TGTTGGAATTTTTTGTGTCTCTCACTCGGAAGGACATATGGGAGGGCAAATCATTTAA
AACATCAGAATGAGTATTTGGTTTAGAGTTTGGCAACATATGCCCATATGCTGGCTGCC
ATGAACAAAGGTTGGCTATAAAGAGGTCATCAGTATATGAAACAGCCCCCTGCTGTCC
ATTCCTTATTCCATAGAAAAGCCTTGACTTGAGGTTAGATTTTTTTTATATTTTGTTTTG
TGTTATTTTTTTCTTTAACATCCCTAAAATTTTCCTTACATGTTTTACTAGCCAGATTTTT
CCTCCTCTCCTGACTACTCCCAGTCATAGCTGTCCCTCTTCTCTTATGGAGATCCCTCG
ACCTGCAGCCCAAGCTTGGCGTAATCATGGTCATAGCTGTTTCCTGTGTGAAATTGTT
ATCCGCTCACAATTCCACACAACATACGAGCCGGAAGCATAAAGTGTAAAGCCTGGG
GTGCCTAATGAGTGAGCTAACTCACATTAATTGCGTTGCGCTCACTGCCCGCTTTCCA
GTCGGGAAACCTGTCGTGCCAGCGGATCCGCATCTCAATTAGTCAGCAACCATAGTCC
CGCCCCTAACTCCGCCCATCCCGCCCCTAACTCCGCCCAGTTCCGCCCATTCTCCGCC
CCATGGCTGACTAATTTTTTTTATTTATGCAGAGGCCGAGGCCGCCTCGGCCTCTGAG
CTATTCCAGAAGTAGTGAGGAGGCTTTTTTGGAGGCCTAGGCTTTTGCAAAAAGCTA
ACTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATC
TTATCATGTCTGGATCCGCTGCATTAATGAATCGGCCAACGCGCGGGGAGAGGCGGTT
TGCGTATTGGGCGCTCTTCCGCTTCCTCGCTCACTGACTCGCTGCGCTCGGTCGTTCG
GCTGCGGCGAGCGGTATCAGCTCACTCAAAGGCGGTAATACGGTTATCCACAGAATC
AGGGGATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCAATGCTCACG
CTGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAG
CGAGGTATGTAGGCGGTGCTACAGAGTTCTTGAAGTGGTGGCCTAACTACGGCTACA
CTAGAAGGACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGTTTTTTTGT
TTGCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGT
GTAGATAACTACGATACGGGAGGGCTTACCATCTGGCCCCAGTGCTGCAATGATACCG
CGAGACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCAT
TGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGC
TCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGTTGC
TCTTGCCCGGCGTCAATACGGGATAATACCGCGCCACATAGCAGAACTTTAAAAGTGC
TCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCA
CCAGCGTTTCTGGGTGAGCAAAAACAGGAAGGCAAAATGCCGCAAAAAAGGGAATA
AGGGCGACACGGAAATGTTGAATACTCATACTCTTCCTTTTTCAATATTATTGAAGCAT
TTATCAGGGTTATTGTCTCATGAGCGGATACATATTTGAATGTATTTAGAAAAATAAAC
AAATAGGGGTTCCGCGCACATTTCCCCGAAAAGTGCCACCTG

Subsequently, a series of expression plasmids of different viral envelope proteins were constructed (pcaggs-CHIKV-E, VEEV-E, EBOV-GP, NIV-GF, LASV-GP, RABV-G, H7N1-HANA, and RSV-F). The sequence of Pcaggs-CHIKV-E is shown in SEQ ID NO: 5, wherein the sequence of the pcaggs vector is shown in SEQ ID NO: 6, and the sequence of CHIKV-E is shown in SEQ ID NO: 7. The construction method is as follows: in the multiple cloning site of the pcaggs vector (preserved in our laboratory), the synthesized CHIKV-E fragment was inserted using NotI and NheI restriction enzyme sites to obtain the Pcaggs-CHIKV-E clone. Other expression plasmids of viral envelope proteins were constructed in the same way. The vector sequence is shown in SEQ ID NO: 6, the VEEV-E sequence is shown in SEQ ID NO: 8, the EBOV-GP sequence is shown in SEQ ID NO: 9, the NIV-GF sequence is shown in SEQ ID NO: 10, the LASV-GP sequence is shown in SEQ ID NO: 11, the RABV-G sequence is shown in SEQ ID NO: 12, the H7N1-HANA sequence is shown in SEQ ID NO: 13, and the RSV-F sequence is shown in SEQ ID NO: 14.

Subsequently, the packaging efficiency and the expression of target proteins of different viral envelope proteins were verified. The specific experimental process is as follows: firstly, 2.5×10⁵ BHK-21 cells (preserved in our laboratory) were inoculated in a 6-well plate and cultured overnight at 37° C. The expression plasmids of corresponding viral envelope proteins were transfected into BHK-21 cells using fugene liposomes. The transfection system consisted of 4 μl fugene liposomes mixed with 1 μg envelope protein expression plasmids. After standing for 15 minutes, the mixture was dropped into the BHK-21 cells cultured overnight. After 12 hours, VEEVrep-EGFP saRNA was transfected using Dmrie-C liposomes. The transfection system was prepared by adding 4 μl Dmrie-C liposomes and 1 μg RNA to 1 ml OPTI-MEM medium. After mixing, the original medium in BHK-21 cells was replaced with this mixture. After 4-6 hours, it was replaced with fresh 2% FBS DMEM medium. The expression of EGFP was observed at different time points after RNA transfection, and the cell culture supernatant was collected at the 48-hour time point, aliquoted and stored at −80° C. Subsequently, the collected 48-hour cell culture supernatant was used to verify the delivery effect on naïve BHK-21 cells, and the expression of EGFP was observed 24 hours after delivery. The specific experimental process is as follows: firstly, 2.5×10⁵ BHK-21 cells were inoculated in a 6-well plate. After overnight culture at 37° C., the original medium was replaced with 2% FBS DMEM medium, and 500 μl of the aforementioned collected 48-hour post-transfection cell culture supernatant was added. The expression of EGFP was observed using a fluorescence microscope after 24 hours.

### Experimental Results

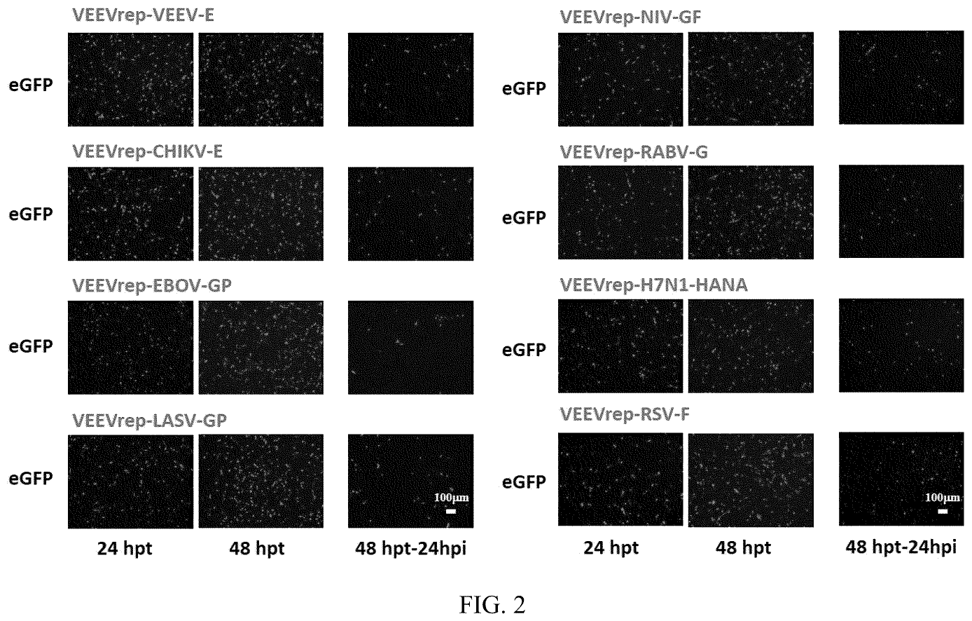

It was found that VEEVrep-EGFP saRNA could effectively express the target protein EGFP after transfecting cells, which proved the ability of the alphavirus genome to self-replicate and express exogenous genes. Further observation showed that the supernatants collected after co-transfection of saRNA with the expression plasmids constructed by the envelope proteins of these 8 different viruses could effectively deliver saRNA and express the target protein EGFP after infecting naïve BHK-21 cells (as shown in FIG. 2). Based on the above results, it is inferred that the co-transfection of VEEVrep-EGFP saRNA with the expression plasmids constructed by the envelope proteins of different viruses can form microvesicles, which can effectively deliver saRNA and express the target protein EGFP. That is, the VRV delivery platform constructed in the present disclosure can achieve effective delivery of saRNA and effective expression of target proteins.

The nucleotide sequence of SEQ ID NO: 6 is as follows:

GTCGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGC
TGACCGCCCAACGACCCCCGCCCATTGACGTCAATAATGACGTATGTTCCCATAGTAA
CGCCAATAGGGACTTTCCATTGACGTCAATGGGTGGACTATTTACGGTAAACTGCCCA
CTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTG
GCAGTACATCTACGTATTAGTCATCGCTATTACCATGGGTCGAGGTGAGCCCCACGTTC
TGCTTCACTCTCCCCATCTCCCCCCCCTCCCCACCCCCAATTTTGTATTTATTTATTTTT
TAATTATTTTGTGCAGCGATGGGGGCGGGGGGGGGGGGGGCGCGCGCCAGGCGGGG
CGGGGGGGGCGAGGGGCGGGGCGGGGCGAGGCGGAGAGGTGCGGCGGCAGCCAA
TCAGAGCGGCGCGCTCCGAAAGTTTCCTTTTATGGCGAGGCGGCGGCGGCGGCGGCC
CTATAAAAAGCGAAGCGCGCGGCGGGCGGGAGTCGCTGCGTTGCCTTCGCCCCGTGC
CCCGCTCCGCGCCGCCTCGCGCCGCCCGCCCCGGCTCTGACTGACCGCGTTACTCCC
ACAGGTGAGCGGGCGGGACGGCCCTTCTCCTCCGGGCTGTAATTAGCGCTTGGTTTA
ATGACGGCTCGTTTCTTTTCTGTGGCTGCGTGAAAGCCTTAAAGGGCTCCGGGAGGG
CCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCG
GGGCTTTGTGCGCTCCGCGTGTGCGCGAGGGGAGCGCGGCCGGGGGCGGTGCCCCG
CGGTGCGGGGGGGCTGCGAGGGGAACAAAGGCTGCGTGCGGGGTGTGTGCGTGGG
GGGGTGAGCAGGGGGTGTGGGCGCGGCGGTCGGGCTGTAACCCCCCCCTGCACCCC
CCTCCCCGAGTTGCTGAGCACGGCCCGGCTTCGGGTGCGGGGCTCCGTGCGGGGCGT
GGCGCGGGGCTCGCCGTGCCGGGCGGGGGGTGGCGGCAGGTGGGGGTGCCGGGCG
GGGCGGGGCCGCCTCGGGCCGGGGAGGGCTCGGGGGAGGGGCGCGGCGGCCCCGG
AGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGG
AGGCGCCGCCGCACCCCCTCTAGCGGGCGCGGGGCGAAGCGGTGCGGCGCCGGCAG
GAAGGAAATGGGCGGGGAGGGCCTTCGTGCGTCGCCGCGCCGCCGTCCCCTTCTCCC
TCTCCAGCCTCGGGGCTGTCCGCGGGGGGACGGCTGCCTTCGGGGGGGACGGGGCA
GGGCGGGGTTCGGCTTCTGGCGTGTGACCGGCGGCTCTAGAGCCTCTGCTAACCATG
TTCATGCCTTCTTCTTTTTCCTACAGCTCCTGGGCAACGTGCTGGTTATTGTGCTGTCT
CATCATTTTGGCAAAGAATTCGGCGGCCGCGCTAGCAGATCTTTTTCCCTCTGCCAAA
AATTATGGGGACATCATGAAGCCCCTTGAGCATCTGACTTCTGGCTAATAAAGGAAAT
TTATTTTCATTGCAATAGTGTGTTGGAATTTTTTGTGTCTCTCACTCGGAAGGACATAT
GGGAGGGCAAATCATTTAAAACATCAGAATGAGTATTTGGTTTAGAGTTTGGCAACAT
ATGCCCATATGCTGGCTGCCATGAACAAAGGTTGGCTATAAAGAGGTCATCAGTATAT
GAAACAGCCCCCTGCTGTCCATTCCTTATTCCATAGAAAAGCCTTGACTTGAGGTTAG
ATTTTTTTTATATTTTGTTTTGTGTTATTTTTTTCTTTAACATCCCTAAAATTTTCCTTACA
TGTTTTACTAGCCAGATTTTTCCTCCTCTCCTGACTACTCCCAGTCATAGCTGTCCCTC
TTCTCTTATGGAGATCCCTCGACCTGCAGCCCAAGCTTGGCGTAATCATGGTCATAGC
TGTTTCCTGTGTGAAATTGTTATCCGCTCACAATTCCACACAACATACGAGCCGGAAG
CATAAAGTGTAAAGCCTGGGGTGCCTAATGAGTGAGCTAACTCACATTAATTGCGTTG
CGCTCACTGCCCGCTTTCCAGTCGGGAAACCTGTCGTGCCAGCGGATCCGCATCTCA
ATTAGTCAGCAACCATAGTCCCGCCCCTAACTCCGCCCATCCCGCCCCTAACTCCGCC
CAGTTCCGCCCATTCTCCGCCCCATGGCTGACTAATTTTTTTTATTTATGCAGAGGCCG
AGGCCGCCTCGGCCTCTGAGCTATTCCAGAAGTAGTGAGGAGGCTTTTTTGGAGGCC
TAGGCTTTTGCAAAAAGCTAACTTGTTTATTGCAGCTTATAATGGTTACAAATAAAGCA
ATAGCATCACAAATTTCACAAATAAAGCATTTTTTTCACTGCATTCTAGTTGTGGTTTG
TCCAAACTCATCAATGTATCTTATCATGTCTGGATCCGCTGCATTAATGAATCGGCCAA
CGCGCGGGGAGAGGCGGTTTGCGTATTGGGCGCTCTTCCGCTTCCTCGCTCACTGAC
TCGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAAACCC
GACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGG
CGCTTTCTCAATGCTCACGCTGTAGGTATCTCAGTTCGGTGTAGGTCGTTCGCTCCAA
GCTGGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCCAC
TGGTAACAGGATTAGCAGAGCGAGGTATGTAGGCGGTGCTACAGAGTTCTTGAAGTG
GTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTAAA
TTAAAAATGAAGTTTTAAATCAATCTAAAGTATATATGAGTAAACTTGGTCTGACAGTT
ACCAATGCTTAATCAGTGAGGCACCTATCTCAGCGATCTGTCTATTTCGTTCATCCATA
GTTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCAAT
AAACCAGCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGT
ATGGCTTCATTCAGCTCCGGTTCCCAACGATCAAGGCGAGTTACATGATCCCCCATGT
TGTGCAAAAAAGCGGTTAGCTCCTTCGGTCCTCCGATCGTTGTCAGAAGTAAGTTGG
CCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACA
TAGCAGAACTTTAAAAGTGCTCATCATTGGAAAACGTTCTTCGGGGCGAAAACTCTC
AAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATACTCTTC
CTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGC
CACCTG

The nucleotide sequence of SEQ ID NO: 7 is as follows:

ATGAGTCTGGCCATTCCAGTTATGTGCCTGCTGGCAAATACCACGTTCCCCTG
CTCCCAGCCCCCTTGCACACCCTGCTGCTACGAAAAAGAGCCGGAGAAAACCTTGC
GCATGCTTGAAGACAATGTCATGAGCCCCGGGTACTATCAGCTGCTACAAGCATCCTT
AACATGTTCTCCCCGACGCCAGCGGCGCAGTATTAAGGACCACTTCAATGTCTATAAA
GCCACAAGACCGTACCTAGCTCACTGTCCCGACTGTGGAGAAGGGCACTCGTGCCAT
AGTCCCGTAGCGCTAGAACGCATCAGAAACGAAGCGACAGACGGGACGTTGAAAAT
CCAGGTTTCCTTGCAAATCGGAATAAAGACGGATGATAGCCATGATTGGACCAAGCTG
CGTTATATGGACAATCACATGCCAGCAGACGCAGAGCGGGCCGGGCTATTTGTAAGA
ACGTCAGCACCGTGCACGATTACTGGAACAATGGGACACTTCATTCTGGCCCGATGT
CCGAAAGGAGAAACTCTGACGGTAGGGTTCACTGACGGTAGGAAGATCAGTCACTC
ATGTACGCACCCATTTCACCATGACCCTCCTGTGATAGGCCGGGAAAAATTCCATTCC
CGACCGCAGCACGGTAGGGAACTACCTTGCAGCACGTACGCGCAGAGCACCGCTGC
AACTGCCGAGGAGATAGAGGTACATATGCCCCCAGACACCCCAGATCGCACATTAATG
TCACAACAGTCCGGCAATGTAAAGATCACAGTCAATAGTCAGACGGTGCGGTACAAG
TGCAATTGTGGTGACTCAAGTGAAGGATTAACCACTACAGATAAAGTGATTAATAACT
GCAAGGTTGATCAATGCCATGCCGCGGTCACCAATCACAAAAAATGGCAGTATAATTC
CCCTCTGGTCCCGCGTAATGCTGAATTCGGGGACCGGAAAGGAAAAGTTCACATTCC
ATTTCCTCTGGCAAATGTGACATGCAGGGTGCCTAAAGCAAGAAACCCCACCGTGAC
GTACGGAAAAAACCAAGTCATCATGTTGCTGTATCCTGACCACCCAACGCTCCTGTCC
TACAGGAATATGGGAGAAGAACCAAACTATCAAGAAGAGTGGGTGACGCATAAGAA
GGAGATCAGGTTAACCGTGCCGACTGAAGGGCTCGAGGTCACGTGGGGTAACAATG
AGCCGTACAAGTATTGGCCGCAGTTATCCACAAACGGTACAGCCCACGGCCACCCGC
ATGAGATAATTCTGTATTATTATGAGCTGTACCCAACTATGACTGTGGTAGTTTTGTCA
GTGGCCTCGTTCATACTCCTGTCGATGGTGGGTGTGGCAGTGGGGATGTGCATGTGTG
CACGACGCAGATGCATTACACCGTACGAACTGACACCAGGAGCTACCGTCCCTTTCC
TGCTTAGCCTAATATGCTGCATTAGAACAGCTAAAGCGGCCACATACCAGGAGGCCGC
GGTATACCTGTGGAACGAGCAGCAGCCTTTATTTTGGCTGCAAGCCCTTATTCCGCTG
GCAGCCCTGATTGTCCTATGTAACTGTCTGAGACTCTTACCATGCTGTTGTAAAATGTT
GACTTTTTTAGCCGTACTGAGCGTCGGTGCCCACACTGTGAGTGCGTACGAACACGT
AACAGTGATCCCGAACACGGTGGGAGTACCGTATAAGACTCTAGTCAACAGACCGGG
CTACAGCCCCATGGTATTGGAGATGGAGCTTCTGTCTGTCACCTTGGAACCAACGCTA
TCGCTTGATTACATCACGTGCGAGTATAAAACCGTTATCCCGTCTCCGTACGTGAAATG
CTGCGGTACAGCAGAGTGTAAGGACAAGAGCCTACCTGATTACAGCTGTAAGGTCTT
CACCGGCGTCTACCCATTCATGTGGGGCGGCGCCTACTGCTTCTGCGACACCGAAAAT
ACGCAATTGAGCGAAGCACATGTGGAGAAGTCCGAATCATGCAAAACAGAATTTGCA
TCAGCATACAGGGCTCATACCGCATCCGCGTCAGCTAAGCTCCGCGTCCTTTACCAAG
GAAATAATATCACTGTAGCTGCTTATGCAAACGGCGACCATGCCGTCACAGTTAAGGA
CGCTAAATTCATAGTGGGGCCAATGTCTTCAGCCTGGACACCTTTCGACAATAAAATC
GTGGTGTACAAAGGCGACGTCTACAACATGGACTACCCGCCCTTCGGCGCAGGAAGA
CCAGGACAATTTGGCGACATCCAAAGTCGCACGCCTGAGAGCGAAGACGTCTATGCT
AATACACAACTGGTACTGCAGAGACCGTCCGCGGGTACGGTGCACGTGCCGTACTCT
CAGGCACCATCTGGCTTCAAGTATTGGCTAAAAGAACGAGGGGCGTCGCTGCAGCAC
ACAGCACCATTTGGCTGTCAAATAGCAACAAACCCGGTAAGAGCGATGAACTGCGCC
GTAGGGAACATGCCTATCTCCATCGACATACCGGACGCGGCCTTTACCAGGGTCGTCG
ACGCGCCATCTTTAACGGACATGTCGTGTGAGGTATCAGCCTGCACCCATTCCTCAGA
CTTTGGGGGCGTAGCCATCATTAAATATGCAGCCAGTAAGAAAGGCAAGTGTGCAGT
GCACTCGATGACTAACGCCGTCACTATTCGGGAAGCTGAAATAGAAGTAGAAGGGAA
CTCTCAGTTGCAAATCTCTTTTTCGACGGCCCTAGCCAGCGCCGAATTTCGCGTACAA
GTCTGTTCTACACAAGTACACTGTGCAGCCGAGTGCCATCCACCGAAAGACCATATAG
TCAATTACCCGGCGTCACACACCACCCTCGGGGTCCAAGACATTTCCGCTACGGCGAT
GTCATGGGTGCAGAAGATCACGGGAGGTGTGGGACTGGTTGTCGCTGTTGCAGCACT
GATCCTAATCGTGGTGCTATGCGTGTCGTTTAGCAGGCACTAA

The nucleotide sequence of SEQ ID NO: 8 is as follows:

ATGTCACTAGTGACCACCATGTGTCTGCTCGCCAATGTGACGTTCCCATGTGC
TCAACCACCAATTTGCTACGACAGAAAACCAGCAGAGACTTTGGCCATGCTCAGCGT
TAACGTTGACAACCCGGGCTACGATGAGCTGCTGGAAGCAGCTGTTAAGTGCCCCGG
AAGGAAAAGGAGATCCACCGAGGAGCTGTTTAATGAGTATAAGCTAACGCGCCCTTA
CATGGCCAGATGCATCAGATGTGCAGTTGGGAGCTGCCATAGTCCAATAGCAATCGAG
GCAGTAAAGAGCGACGGGCACGACGGTTATGTTAGACTTCAGACTTCCTCGCAGTAT
GGCCTGGATTCCTCCGGCAACTTAAAGGGCAGGACCATGCGGTATGACATGCACGGG
ACCATTAAAGAGATACCACTACATCAAGTGTCACTCTATACATCTCGCCCGTGTCACAT
TGTGGATGGGCACGGTTATTTCCTGCTTGCCAGGTGCCCGGCAGGGGACTCCATCAC
CATGGAATTTAAGAAAGATTCCGTCAGACACTCCTGCTCGGTGCCGTATGAAGTGAA
ATTTAATCCTGTAGGCAGAGAACTCTATACTCATCCCCCAGAACACGGAGTAGAGCAA
GCGTGCCAAGTCTACGCACATGATGCACAGAACAGAGGAGCTTATGTCGAGATGCAC
CTCCCGGGCTCAGAAGTGGACAGCAGTTTGGTTTCCTTGAGCGGCAGTTCAGTCACC
GTGACACCTCCTGATGGGACTAGCGCCCTGGTGGAATGCGAGTGTGGCGGCACAAA
GATCTCCGAGACCATCAACAAGACAAAACAGTTCAGCCAGTGCACAAAGAAGGAGC
AGTGCAGAGCATATCGGCTGCAGAACaATAAGTGGGTGTATAATTCTGACAAACTGCC
CAAAGCAGCGGGAGCCACCTTAAAAGGAAAACTGCATGTCCCATTCTTGCTGGCAGA
CGGCAAATGCACCGTGCCTCTAGCACCAGAACCTATGATAACCTTCGGTTTCAGATCA
GTGTCACTGAAACTGCACCCTAAGAATCCCACATATCTAAcCACCCGCCAACTTGCTG
ATGAGCCTCACTACACGCACGAGCTCATATCTGAACCAGCTGTTAGGAATTTTACCGT
CACCGAAAAAGGGTGGGAGTTTGTATGGGGAAACCACCCGCCGAAAAGGTTTTGGG
CACAGGAAACAGCACCCGGAAATCCACATGGGCTACCGCACGAGGTGATAACTCATT
ATTACCACAGATACCCTATGTCCACCATCCTGGGTTTGTCAATTTGTGCCGCCATTGCA
ACCGTTTCCGTTGCAGCGTCTACCTGGCTGTTTTGCAGATCTAGAGTTGCGTGCCTAA
CTCCTTACCGGCTAACACCTAACGCTAGGATACCATTTTGTCTGGCTGTGCTTTGCTGC
GCCCGCACTGCCCGGGCCGAGACCACCTGGGAGTCCTTGGATCACCTATGGAACAAT
AACCAACAGATGTTCTGGATTCAATTGCTGATCCCTCTGGCCGCCTTGATCGTAGTGA
CTCGCCTGCTCAGGTGCGTGTGCTGTGTCGTGCCTTTTTTAGTCATGGCCGGCGCCGC
AGGCGCCGGCGCCTACGAGCACGCGACCACGATGCCGAGCCAAGCGGGAATCTCGT
ATAACACTATAGTCAACAGAGCAGGCTACGCACCACTCCCTATCAGCATAACACCAAC
AAAGATCAAGCTGATACCTACAGTGAACTTGGAGTACGTCACCTGCCACTACAAAAC
AGGAATGGATTCACCAGCCATCAAATGCTGCGGATCTCAGGAATGCACTCCAACTTAC
AGGCCTGATGAACAGTGCAAAGTCTTCACAGGGGTTTACCCGTTCATGTGGGGTGGT
GCATATTGCTTTTGCGACACTGAGAACACCCAAGTCAGCAAGGCCTACGTAATGAAA
TCTGACGACTGCCTTGCGGATCATGCTGAAGCATATAAAGCGCACACAGCCTCAGTG
CAGGCGTTCCTCAACATCACAGTGGGAGAACACTCTATTGTGACTACCGTGTATGTGA
ATGGAGAAACTCCTGTGAATTTCAATGGGGTCAAAATAACTGCAGGTCCGCTTTCCA
CAGCTTGGACACCCTTTGATCGCAAAATCGTGCAGTATGCCGGGGAGATCTATAATTA
TGATTTTCCTGAGTATGGGGCAGGACAACCAGGAGCATTTGGAGATATACAATCCAGA
ACAGTCTCAAGCTCTGATCTGTATGCCAATACCAACCTAGTGCTGCAGAGACCCAAA
GCAGGAGCGATCCACGTGCCATACACTCAGGCACCTTCGGGTTTTGAGCAATGGAAG
AAAGATAAAGCTCCATCATTGAAATTTACCGCCCCTTTCGGATGCGAAATATATACAAA
CCCCATTCGCGCCGAAAACTGTGCTGTAGGGTCAATTCCATTAGCCTTTGACATTCCC
GACGCCTTGTTCACCAGGGTGTCAGAAACACCGACACTTTCAGCGGCCGAATGCACT
CTTAACGAGTGCGTGTATTCTTCCGACTTTGGTGGGATCGCCACGGTCAAGTACTCGG
CCAGCAAGTCAGGCAAGTGCGCAGTCCATGTGCCATCAGGGACTGCTACCCTAAAAG
AAGCAGCAGTCGAGCTAACCGAGCAAGGGTCGGCGACTATCCATTTCTCGACCGCAt
ATATCCACCCGGAGTTCAGGCTCCAAATATGCACATCATATGTTACGTGCAAAGGTGAT
TGTCACCCCCCGAAAGACCATtTTGTGACACACCCTCAGTATCACGCCCAAACATTTA
CAGCCGCGGTGTCAAAAACCGCGTGGACGTGGTTAACATCCCTGCTGGGAGGATCAG
CCGTAATTATTATAATTGGCTTGGTGCTGGCTACTATTGTGGCCATGTACGTGCTGACC
AACCAGAAACATAATTGA

The nucleotide sequence of SEQ ID NO: 9 is as follows:

ATGggcgttacaggaatattgcagttacctcgtgatcgattcaagaggacatcattctttctttgggtaattatccttttccaaaga
acattttccatcccacttggagtcatccacaatagcacattacaggttagtgatgtcgacaaactagtttgtcgtgacaaactgtcatcca
caaatcaattgagatcagttggactgaatctcgaagggaatggagtggcaactgacgtgccatctgcaactaaaagatggggcttcaggtcc
ggtgtcccaccaaaggtggtcaattatgaagctggtgaatgggctgaaaactgctacaatcttgaaatcaaaaaacctgacgggagtgagtg
tctaccagcagcgccagacgggattcggggcttcccccggtgccggtatgtgcacaaagtatcaggaacgggaccgtgtgccggagactttg
ccttccataaagagggtgctttcttcctgtatgatcgacttgcttccacagttatctaccgaggaacgactttcgctgaaggtgtcgttgca
tttctgatactgccccaagctaagaaggacttcttcagctcacaccccttgagagagccggtcaatgcaacggaggacccgtctagtggcta
ctattctaccacaattagatatcaggctaccggttttggaaccaatgagacagagtacttgttcgaggttgacaatttgacctacgtccaac
ttgaatcaagattcacaccacagtttctgctccagctgaatgagacaatatatacaagtgggaaaaggagcaataccacgggaaaactaatt
tggaaggtcaaccccgaaattgatacaacaatcggggagtgggccttctgggaaactaaaaaaAacctcactagaaaaattcgcagtgaaga
gttgtctttcacagttgtatcaaacggagccaaaaacatcagtggtcagagtccggcgcgaacttcttccgacccagggaccaacacaacaa
ctgaagaccacaaaatcatggcttcagaaaattcctctgcaatggttcaagtgcacagtcaaggaagggaagctgcagtgtcgcatctaaca
acccttgccacaatctccacgagtccccaatccctcacaaccaaaccaggtccggacaacagcacccataatacacccgtgtataaacttga
catctctgaggcaactcaagttgaacaacatcaccgcagaacagacaacgacagcacagcctccgacactccctctgccacgaccgcagccg
gacccccaaaagcagagaacaccaacacgagcaagagcactgacttcctggaccccgccaccacaacaagtccccaaaaccacagcgaga
ccgctggcaacaacaacactcatcaccaagataccggagaagagagtgccagcagcgggaagctaggcttaattaccaatactattgctg
gagtcgcaggactgatcacaggcgggagaagaactcgaagagaagcaattgtcaatgctcaacccaaatgcaaccctaatttacattactg
gactactcaggatgaaggtgctgcaatcggactggcctggataccatatttcgggccagcagccgagggaatttacatagaggggctaatg
cacaatcaagatggtttaatctgtgggttgagacagctggccaacgagacgactcaagctcttcaactgttcctgagagccacaactgagct
acgcaccttttcaatcctcaaccgtaaggcaattgatttcttgctgcagcgatggggcggcacatgccacattctgggaccggactgctgta
tcgaaccacatgattggaccaagaacataacagacaaaattgatcagattattcatgattttgttgataaaacccttccggaccagggggac
aatgacaattggtggacaggatggagacaatggataccggcaggtattggagttacaggcgttataattgcagttatcgctttattctgtat
atgcaaatttgtcttttagttaattaa

The nucleotide sequence of SEQ ID NO: 10 is as follows:

atgccggcagaaaacaagaaagttagattcgaaaatactacttcagacaaagggaaaattcctagtaaagttattaagagctact
acggaaccatggacattaagaaaataaatgaaggattattggacagcaaaatattaagtgctttcaacacagtaatagcattgcttggatctat
cgtgatcatagtgatgaatataatgatcatccaaaattacacaagatcaacagacaatcaggccgtgatcaaagatgcgttgcagggtatcca
acagcagatcaaagggcttgctgacaaaatcggcacagagatagggcccaaagtatcactgattgacacatccagtaccattactatccca
gctaacattgggctgttaggttcaaagatcagccagtcgactgcaagtataaatgagaatgtgaatgaaaaatgcaaattcacactgcctccc
ttgaaaatccacgaatgtaacatttcttgtcctaacccactcccttttagagagtataggccacagacagaaggggtgagcaatctagtaggat
tacctaataatatttgcctgcaaaagacatctaatcagatattgaagccaaagctgatttcatacactttacccgtagtcggtcaaagtggtacct
gtatcacagacccattgctggctatggacgagggctattttgcatatagccacctggaaagaatcggatcatgttcaagaggggtctccaaac
aaagaataataggagttggagaggtactagacagaggtgatgaagttccttctttatttatgaccaatgtctggaccccaccaaatccaaaca
ccgtttaccactgtagtgctgtatacaacaatgaattctattatgtactttgtgcagtgtcaactgttggagaccctattctgaatagcacctact
ggtccggatctctaatgatgacccgtctagctgtgaaacccaagagtaatggtgggggttacaatcaacatcaacttgccctacgaagtatcga
gaaagggaggtatgataaagttatgccgtatggaccttcaggcatcaaacagggtgacaccctgtattttcctgctgtaggatttttggtcagg
acagagtttaaatacaatgattcaaattgtcccatcacgaagtgtcaatacagtaaacctgaaaattgcaggctatctatggggattagaccaa
acagccattatatccttcgatctggactattaaaatacaatctatcagatggggagaaccccaaagttgtattcattgaaatatctgatcaaagat
tatctattggatctcctagcaaaatctatgattctttgggtcaacctgttttctaccaagcgtcattttcatgggatactatgattaaatttggag
atgttctaacagtcaaccctctggttgtcaattggcgtaataacacggtaatatcaagacccgggcaatcacaatgccctagattcaatacatgtc
cagagatctgctgggaaggagtttataatgatgcattcctaattgacagaatcaattggataagcgcgggtgtattccttgacagcaatcagacc
gcagaaaatcctgtttttactgtattcaaagataatgaaatactttatagggcacaactggcttctgaggacaccaatgcacaaaaaacaataa
ctaattgttttctcttgaagaataagatttggtgcatatcattggttgagatatatgacacaggagacaatgtcataagacccaaactattcgcgg
ttaagataccagagcaatgtacataattaattaatacctgagaggggcccctataactctctacggctaacctgaatggactacgacatAGT
CTAGTCCGCCAAGaccatggtagttatacttgacaagagatgttattgtaatcttttaatattgattttgatgatctcggagtgtagtgtt
gggattctacattatgagaaattgagtaaaattggacttgtcaaaggagtaacaagaaaatacaagattaaaagcaatcctctcacaaaagac
attgttataaaaatgattccgaatgtgtcgaacatgtctcagtgcacagggagtgtcatggaaaattataaaacacgattaaacggtatcttaac
acctataaagggagcgttagagatctacaaaaacaacactcatgaccttgtcggtgatgtgagattagccggagttataatggcaggagttg
ctattgggattgcaaccgcagctcaaatcactgcaggtgtagcactatatgaggcaatgaagaatgctgacaacatcaacaaactcaaaagc
agcattgaatcaactaatgaagctgtcgttaaacttcaagagactgcagaaaagacagtctatgtgctgactgctctacaggattacattaata
ctaatttagtaccgacaattgacaagataagctgcaaacagacagaactctcactagatctggcattatcaaagtacctctctgatttgctttttg
tatttggccccaaccttcaagacccagtttctaattcaatgactatacaggctatatctcaggcattcggtggaaattatgaaacactgctaagaa
cattgggttacgctacagaagactttgatgatcttctagaaagtgacagcataacaggtcaaatcatctatgttgatctaagtagctactatataa
ttgtcagggtttattttcctattctgactgaaattcaacaggcctatatccaagagttgttaccagtgagcttcaacaatgataattcagaatgga
tcagtattgtcccaaatttcatattggtaaggaatacattaatatcaaatatagagattggattttgcctaattacaaagaggagcgtgatctgca
accaagattatgccacacctatgaccaacaacatgagagaatgtttaacgggatcgactgagaagtgtcctcgagagctggttgtttcatcaca
tgttcccagatttgcactatctaacggggttctgtttgccaattgcataagtgttacatgtcagtgtcaaacaacaggcagggcaatctcacaat
caggagaacaaactctgctgatgattgacaacaccacctgtcctacagccgtactcggtaatgtgattatcagcttagggaaatatctggggt
cagtaaattataattctgaaggcattgctatcggtcctccagtctttacagataaagttgatatatcaagtcagatatccagcatgaatcagtcct
tacaacagtctaaggactatatcaaagaggctcaacgactccttgatactgttaatccatcattaataagcatgttgtctatgatcatactgtatg
tattatcgatcgcatcgttgtgtatagggttgattacatttatcagttttatcattgttgagaaaaagagaaacacctacagcagattagaggata
ggagagtcagacctacaagcagtggggatctctactacattgggacatag

The nucleotide sequence of SEQ ID NO: 11 is as follows:

ATGGGACAAATAGTGACATTCTTCCAGGAAGTGCCTCATGTAATAGAAGAGG
TGATGAACATTGTTCTCATTGCACTGTCTGTACTAGCAGTGCTGAAAGGTCTGTACAA
TTTTGCAACGTGTGGCCTTGTTGGTTTGGTCACTTTCCTCCTGTTGTGTGGTAGGTCTT
GCACAACCAGTCTTTATAAAGGGGTTTATGAGCTTCAGACTCTGGAACTAAACATGGA
GACACTCAATATGACCATGCCTCTCTCCTGCACAAAGAACAACAGTCATCATTATATA
ATGGTGGGCAATGAGACAGGACTAGAACTGACCTTGACCAACACGAGCATTATTAAT
CACAAATTTTGCAATCTGTCTGATGCCCACAAAAAGAACCTCTATGACCACGCTCTTA
TGAGCATAATCTCAACTTTCCACTTGTCCATCCCCAACTTCAATCAGTATGAGGCAATG
AGCTGCGATTTTAATGGGGGAAAGATTAGTGTGCAGTACAACCTGAGTCACAGCTATG
CTGGGGATGCAGCCAACCATTGTGGTACTGTTGCAAATGGTGTGTTACAGACTTTTAT
GAGGATGGCTTGGGGTGGGAGCTACATTGCTCTTGACTCAGGCCGTGGCAACTGGGA
CTGTATTATGACTAGTTATCAATATCTGATAATCCAAAATACAACCTGGGAAGATCACT
GCCAATTCTCGAGACCATCTCCCATCGGTTATCTCGGGCTCCTCTCACAAAGGACTAG
AGATATTTATATTAGTAGAAGATTGCTAGGCACATTCACATGGACACTGTCAGATTCTG
AAGGTAAAGACACACCAGGGGGATATTGTCTGACCAGGTGGATGCTAATTGAGGCTG
AACTAAAATGCTTCGGGAACACAGCTGTGGCAAAATGTAATGAGAAGCATGATGAGG
AATTTTGTGACATGCTGAGGCTGTTTGACTTCAACAAACAAGCCATTCAAAGGTTGA
AAGCTGAAGCACAAATGAGCATTCAGTTGATCAACAAAGCAGTAAATGCTTTGATAA
ATGACCAACTTATAATGAAGAACCATCTACGGGACATCATGGGAATTCCATACTGTAAT
TACAGCAAGTATTGGTACCTCAACCACACAACTACTGGGAGAACATCACTGCCCAAA
TGTTGGCTTGTATCAAATGGTTCATACTTGAACGAGACCCACTTTTCTGATGATATTGA
ACAACAAGCTGACAATATGATCACTGAGATGTTACAGAAGGAGTATATGGAGAGGCA
GGGGAAGACACCATTGGGTCTAGTTGACCTCTTTGTGTTCAGTACAAGTTTCTATCTT
ATTAGCATCTTCCTTCACCTAGTCAAAATACCAACTCATAGGCATATTGTAGGCAAGTC
GTGTCCCAAACCTCACAGATTGAATCATATGGGCATTTGTTCCTGTGGACTCTACAAA
CAGCCTGGTGTGCCTGTGAAATGGAAGAGATGA

The nucleotide sequence of SEQ ID NO: 12 is as follows:

ATGGTTCCTCAGGCTCTCCTGTTTGTACCCCTTCTGGTTTTTCCATTGTGTTTT
GGGAAATTCCCTATTTACACGATACCAGACAAGCTTGGTCCCTGGAGTCCGATTGACA
TACATCACCTCAGCTGCCCAAACAATTTGGTAGTGGAGGACGAAGGATGCACCAACC
TGTCAGGGTTCTCCTACATGGAACTTAAAGTTGGATACATCTTAGCCATAAAAGTGAA
CGGGTTCACTTGCACAGGCGTTGTGACGGAGGCTGAAACCTACACTAACTTCGTTGG
TTATGTCACAACCACGTTCAAAAGAAAGCATTTCCGCCCAACACCAGATGCATGTAG
AGCCGCGTACAACTGGAAGATGGCCGGTGACCCCAGATATGAAGAGTCTCTACACAA
TCCGTACCCTGACTACCGCTGGCTTCGAACTGTAAAAACCACCAAGGAGTCTCTCGT
TATCATATCTCCAAGTGTGGCAGATTTGGACCCATATGACAGATCCCTTCACTCGAGG
GTCTTCCCTAGCGGGAAGTGCTCAGGAGTAGCGGTGTCTTCTACCTACTGCTCCACTA
ACCACGATTACACCATTTGGATGCCCGAGAATCCGAGACTAGGGATGTCTTGTGACAT
TTTTACCTCCAGTAGAGGGAAGAGAGCATCCAAAGGGAGTGAGACTTGCGGCTTTGT
AGATGAAAGAGGCCTATATAAGTCTTTAAAAGGAGCATGCAAACTCAAGTTATGTGG
AGTTCTAGGACTTAGACTTATGGATGGAACATGGGTCTCGATGCAAACATCAAATGAA
ACCAAATGGTGCCCTCCCGATAAGTTGGTGAACCTGCACGACTTTCGCTCAGACGAA
ATTGAGCACCTTGTTGTAGAGGAGTTGGTCAGGAAGAGAGAGGAGTGTCTGGATGCA
CTAGAGTCCATCATGACAACCAAGTCAGTGAGTTTCAGACGTCTCAGTCATTTAAGA
AAACTTGTCCCTGGGTTTGGAAAAGCATATACCATATTCAACAAGACCTTGATGGAAG
CCGATGCTCACTACAAGTCAGTCGAAACTTGGAATGAGATCCTCCCTTCAAAAGGGT
GTTTAAGAGTTGGGGGGAGGTGTCATCCTCATGTGAACGGGGTGTTTTTCAATGGTAT
AATATTAGGACCTGACGGCAATGTCTTAATCCCAGAGATGCAATCATCCCTCCTCCAG
CAACATATGGAGTTGTTGGAATCCTCGGTTATCCCCCTTGTGCACCCCCTGGCAGACC
CGTCTACCGTTTTCAAGGACGGTGACGAGGCTGAGGATTTTGTTGAAGTTCACCTTCC
CGATGTGCACAATCAGGTCTCAGGAGTTGACTTGGGTCTCCCGAACTGGGGGAAGTA
TGTATTACTGAGTGCAGGGGCCCTGACTGCCTTGATGTTGATAATTTTCCTGATGACAT
GTTGTAGAAGAGTCAATCGATCAGAACCTACGCAACACAATCTCAGAGGGACAGGG
AGGGAGGTGTCAGTCACTCCCCAAAGCGGGAAGATCATATCTTCATGGGAATCACAC
AAGAGTGGGGGTGAGACCAGACTG

The nucleotide sequence of SEQ ID NO: 13 is as follows:

atgaatccaaatcagaagatagtgaccattggatcaatctgtatggtaagtgggataattagcttgatgttacagattgggaacat
aatctcaatatgggtcagtcattcaattcaaacagggaatcaacaccaaactgaaccaatcagaaatactaatttcctcactgagaacactg
tagcttcagtaacattagctggcaattcatctctttgccccatcaaaggatgggctgtacacagtaaagacaacagtataaggattgggtcc
aagggggatgtgtttgtaattagagagccgttcatatcatgctcccatctggaatgcagaaccttctttttgactcagggagctttactgaa
tgacaagcactccaacgggactttcaaagataggagccctcatagaacgctaatgagttgccctataggtgaggctccctctccatataact
cgaggtttgagtctgttgcttggtcggcaagtgcttgccatgatggcaccagttggttgataattggaatttctggtccagacaatggggct
gtggcggtattgaaatacaatggcataataacagacactatcaagagttggaggaataacatactgaggacccaagagtctgaatgtgcatg
tatgaatggctcttgctttactgttatgacagatggaccaagtaatgggcaggcatcatataagattttcaaaatagaaaaagggaaagtag
ttaagtcagtcgaattgaatgcccccaattatcactatgaggaatgctcctgttatcctgaggctggcgaaatcatatgtgtgtgcagggat
aattggcatggctcaaacaggccatgggtatctttcaatcagaatttggagtatcaaataggatatatttgcagtggagttttcggtgacaa
tccacggccgaatgacggtacagggagttgtggaccagtgtcctctaacggggcatatggggtaaaagggttctcatttaaatatggcaatg
gtgtctggatcgggaggaccaaaagcactcattccaggagcggctttgaaatgatttgggatccaaacgggtggactggaacggacagtgag
ttctcgatgaaacaagatatagtagcaataactgattggtcaggatatagcgggagttttgtccagcacccagaactgacaggattggattg
catgagaccttgcttctgggttgagttaatcagagggcggcccaaagagagcacaatttggactagtgggagcagcatatctttttgtggtg
taaatagtgacactgtgagttggtcttggccagacggtgctgagttgccattcaccattgacaagtag

The nucleotide sequence of SEQ ID NO: 14 is as follows:

atggagttgctaatcctcaaagcaaatgcaattaccacaatcctcactgcagtcacattttgttttgcttctggtcaaaacatcactg
aagaattttatcaatcaacatgcagtgcagttagcaaaggctatcttagtgctctgagaactggttggtataccagtgttataactatagaa
ttaagtaatatcaagaaaaataagtgtaatggaacagatgctaaggtaaaattgataaaacaagaattagataaatataaaaatgctgtaac
agaattgcagttgctcatgcaaagcacacaagcaacaaacaatcgagccagaagagaactaccaaggtttatgaattatacactcaacaatg
ccaaaaaaaccaatgtaacattaagcaagaaaaggaaaagaagatttcttggttttttgttaggtgttggatctgcaatcgccagtggcgtt
gctgtatctaaggtcctgcacctagaaggggaagtgaacaagatcaaaagtgctctactatccacaaacaaggctgtagtcagcttatcaaa
tggagttagtgttttaaccagcaaagtgttagacctcaaaaactatatagataaacaattgttacctattgtgaacaagcaaagctgcagca
tatcaaatatagaaactgtgatagagttccaacaaaagaacaacagactactagagattaccagggaatttagtgttaatgcaggcgtaact
acacctgtaagcacttacatgttaactaatagtgaattattgtcattaatcaatgatatgcctataacaaatgatcagaaaaagttaatgtc
caacaatgttcaaatagttagacagcaaagttactctatcatgtccataataaaagaggaagtcttagcatatgtagtacaattaccactat
atggtgttatagatacaccctgttggaaactacacacatcccctctatgtacaaccaacacaaaagaagggtccaacatctgtttaacaaga
actgacagaggatggtactgtgacaatgcaggatcagtatctttcttcccacaagctgaaacatgtaaagttcaatcaaatcgagtatttt
gtgacacaatgaacagtttaacattaccaagtgaagtaaatctctgcaatgttgacatattcaaccccaaatatgattgtaaaattatgact
tcaaaaacagatgtaagcagctccgttatcacatctctaggagccattgtgtcatgctatggcaaaactaaatgtacagcatccaataaaa
atcgtggaatcataaagacattttctaacgggtgcgattatgtatcaaataaaggggtggacactgtgtctgtaggtaacacattatattat
gtaaataagcaagaaggtaaaagtctctatgtaaaaggtgaaccaataataaatttctatgacccattagtattcccctctgatgaatttga
tgcatcaatatctcaagtcaacgagaagattaaccagagcctagcatttattcgtaaatccgatgaattattacataatgtaaatgctggt
aaatccaccacaaatatcatgataactactataattatagtgattatagtaatattgttatcattaattgctgttggactgctcttatactg
taaggccagaagcacaccagtcacactaagcaaagatcaactgagtggtataaataatattgcatttagtaactaa

### Example 3: Cellular Secretion Morphology of Different Viral Envelope Microvesicles Constructed Via the VRV System

In order to further determine the properties of microvesicles obtained from the VRV delivery system, we performed electron microscopic observation on different cells secreting microvesicles which are constructed in Example 2. Following the experimental method in Example 2, we co-transfected VEEVrep-EGFP saRNA with expression plasmids constructed by envelope proteins of different viruses. Cells at 48 hours post-transfection were washed 1-2 times with PBS, fixed with 2.5% glutaraldehyde at room temperature for approximately 4 hours, scraped off with a cell scraper, and transferred to Ep tubes using a pipette. Cells or tissues fixed with glutaraldehyde were rinsed 3 times with 0.1 M phosphate buffer (pH 7.4), 15 minutes each time, then fixed at 20° C. for 2-3 hours with 1% osmic acid pre-cooled at 4° C. (the duration was adjusted based on different samples). They were then rinsed 3 times with 0.1 M phosphate buffer (pH 7.4), 15 minutes each time. Samples were dehydrated with a graded ethanol series (30%, 50%, 70%, 80%, 85%, 90%, 95%, and 100% twice), followed by penetration using a penetrant. The penetrated samples were placed in embedding molds, supplemented with the embedding medium epoxy resin, and polymerized in a 60° C. incubator for 48 hours. Ultrathin sections were then performed, with the section thickness typically ranging from 80 to 100 nm.

### Experimental Results

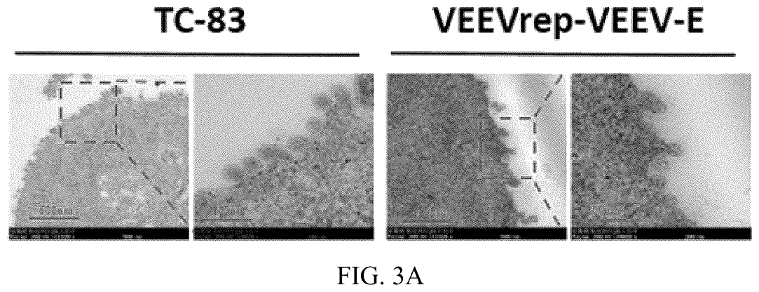

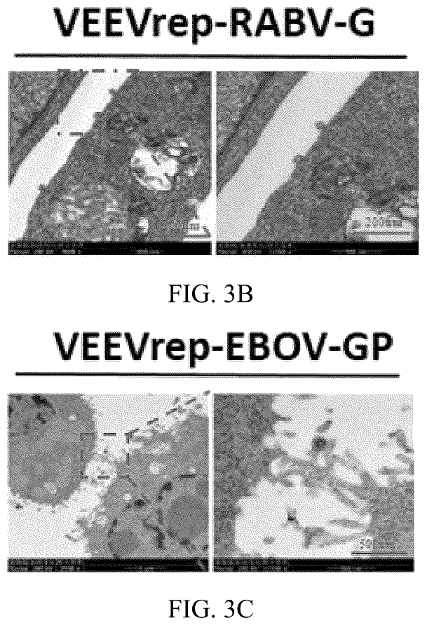

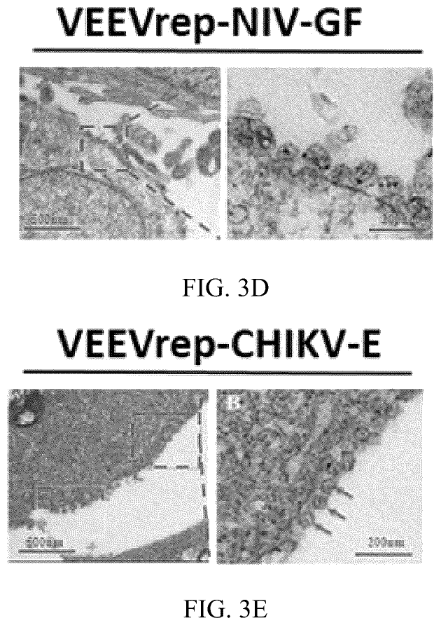

The results showed that viral particles and microvesicle particles were observed on the surfaces of cells infected with wild-type virus TC-83 and VEEVrep-VEEV-E microvesicles, respectively (see FIG. 3A). Among these, the particle size of VEEVrep-VEEV-E microvesicles was similar to that of wild-type virus TC-83, but due to the lack of capsid proteins, VEEV-delC particles lacked a core region similar to that of TC-83 in the center, and their electron density was significantly lower than that of TC-83 virus (FIG. 3A; black arrow indicates the TC-83 core region). Similarly, VEEVrep-RABV-G microvesicles assembled from VEEV replicon-rabies virus envelope glycoprotein G showed clear electron microscopic structures, but their morphology, shape and size differed significantly from those of wild-type RABV (see FIG. 3B). In contrast, electron microscopic structures of VEEVrep-EBOV-GP microvesicles (see FIG. 3C) and VEEVrep-NIV-GF microvesicles (see FIG. 3D) were similar to those of wild-type viruses. Consistent with VEEVrep-VEEV-E, VEEVrep-CHIKV-E microvesicles showed clear electron microscopic structures with a size similar to that of wild-type but lacked a core region formed by capsid proteins (see FIG. 3E). That is, the present disclosure observed the morphological structures of various VRVs constructed by the VRV delivery platform via section electron microscopy and found that they all lack nucleocapsid structures formed by capsid proteins.

### Example 4: Negative Staining Electron Microscopic Structure and Particle Size Observation of VEEVrep-VEEV-E Microvesicles Obtained Via the VRV System

In order to further observe the properties of microvesicles obtained via the VRV delivery system, we mass-produced VEEVrep-VEEV-E microvesicles, which exhibited higher delivery efficiency among them. The collected culture supernatant was then concentrated using PEG8000 and subjected to sucrose gradient density centrifugation, and microvesicles were collected at the corresponding bands. Desugaring was performed using Amicon® Ultra-0.5 ultrafiltration tubes, and the resulting pure microvesicle solution was used for subsequent observation by negative staining electron microscopy (TEM) and particle size detection by dynamic light scattering (DLS).

### Experimental Results

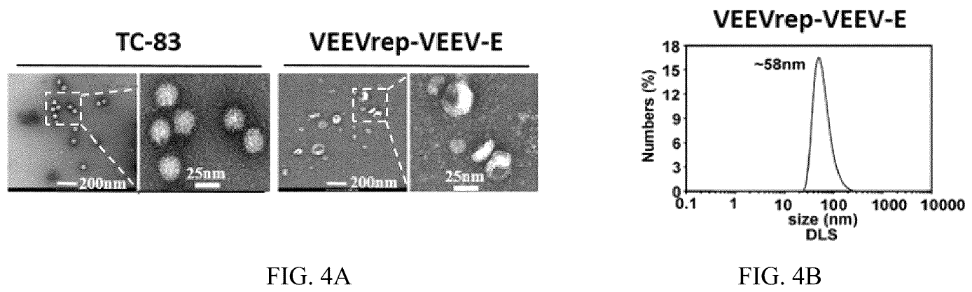

As shown in FIG. 4A, under a 120 kV transmission electron microscope, TC-83 exhibited a regular morphology with a compact core, consistent with literature reports. However, VEEVrep-VEEV-E microvesicles showed irregular morphology with a structure similar to cellular microvesicles (FIG. 4A). Additionally, DLS results showed that the average size of VEEVrep-VEEV-E microvesicles was approximately 58 nm (FIG. 4B). That is, the morphological structure of VEEVrep-VEEV-E microvesicles constructed via the VRV delivery platform in the present disclosure differed significantly from that of wild-type virus, with irregular morphology due to the lack of capsid proteins. However, DLS data showed a relatively concentrated particle size distribution.

### Example 5: Negative Staining Electron Microscopic Structure and Particle Size Observation of VEEVrep-hACE2-E Microvesicles Obtained Via the VRV System

In the preceding Examples, the present disclosure verified the feasibility of the VRV delivery system for delivering target proteins via saRNA using green fluorescent protein (EGFP). In order to enhance its application potential, this Example replaced EGFP with a therapeutic functional gene and observed the expression of the functional gene.

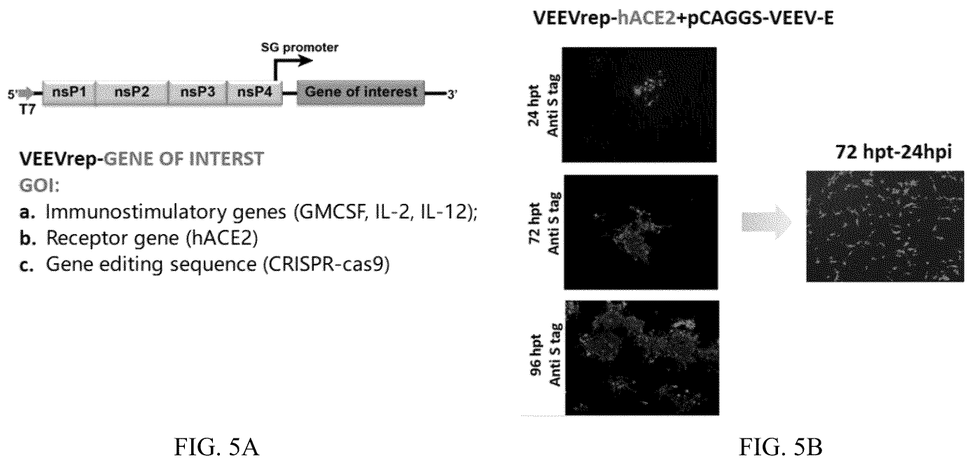

Optional types of target proteins include, but are not limited to: immunostimulatory genes, receptor genes, suicide genes, gene editing sequences, etc., (see FIG. 5A). Furthermore, we selected the novel coronavirus receptor hACE2 as the target gene to test the delivery capability of the VRV system. The hACE2 sequence is shown in SEQ ID NO: 15. The specific experimental process was as follows: firstly, the EGFP gene in the VEEVrep-EGFP saRNA plasmid was replaced with the hACE2 gene through AscI and PacI restriction sites, and in the same way, the corresponding RNA was obtained by reverse transcription. The pcaggs-VEEV-E plasmid and VEEVrep-hACE2-E RNA were transfected according to the method described in Example 2. After co-transfection, the expression of the target proteins at different time points was detected using anti-hACE2 antibodies. Similarly, the supernatant at 48 hours post-infection was collected and infected naïve BHTK-21 cells to test the delivery efficiency.

### Experimental Results

HACE2 expression was detected by IFA. As shown in FIG. 5B, hACE2 expression could be detected at 24 hours post-transfection. Its expression increases over time, peaking at the 96-hour time point (see FIG. 5B, left). Supernatant at 72 hours post-infection was collected and infected naïve BHlK cells, and hACE2 expression was detected at 24 hpi. Results showed that hACE2 was highly expressed with an infectious positive rate of nearly 500% (see FIG. 5B, right). It demonstrates that the VRV delivery system has excellent capability for delivering target genes.

The nucleotide sequence of SEQ ID NO: 15 is as follows:

ATGTCAAGCTCTTCCTGGCTCCTTCTCAGCCTTGTTGCTGTAACTGCTGCTCA
GTCCACCATTGAGGAACAGGCCAAGACATTTTTGGACAAGTTTAACCACGAAGCCGA
AGACCTGTTCTATCAAAGTTCACTTGCTTCTTGGAATTATAACACCAATATTACTGAAG
AGAATGTCCAAAACATGAATAATGCTGGGGACAAATGGTCTGCCTTTTTAAAGGAAC
AGTCCACACTTGCCCAAATGTATCCACTACAAGAAATTCAGAATCTCACAGTCAAGCT
TCAGCTGCAGGCTCTTCAGCAAAATGGGTCTTCAGTGCTCTCAGAAGACAAGAGCAA
ACGGTTGAACACAATTCTAAATACAATGAGCACCATCTACAGTACTGGAAAAGTTTGT
AACCCAGATAATCCACAAGAATGCTTATTACTTGAACCAGGTTTGAATGAAATAATGG
CAAACAGTTTAGACTACAATGAGAGGCTCTGGGCTTGGGAAAGCTGGAGATCTGAGG
TCGGCAAGCAGCTGAGGCCATTATATGAAGAGTATGTGGTCTTGAAAAATGAGATGG
CAAGAGCAAATCATTATGAGGACTATGGGGATTATTGGAGAGGAGACTATGAAGTAA
ATGGGGTAGATGGCTATGACTACAGCCGCGGCCAGTTGATTGAAGATGTGGAACATAC
CTTTGAAGAGATTAAACCATTATATGAACATCTTCATGCCTATGTGAGGGCAAAGTTG
ATGAATGCCTATCCTTCCTATATCAGTCCAATTGGATGCCTCCCTGCTCATTTGCTTGGT
GATATGTGGGGTAGATTTTGGACAAATCTGTACTCTTTGACAGTTCCCTTTGGACAGA
AACCAAACATAGATGTTACTGATGCAATGGTGGACCAGGCCTGGGATGCACAGAGAA
TATTCAAGGAGGCCGAGAAGTTCTTTGTATCTGTTGGTCTTCCTAATATGACTCAAGG
ATTCTGGGAAAATTCCATGCTAACGGACCCAGGAAATGTTCAGAAAGCAGTCTGCCA
TCCCACAGCTTGGGACCTGGGGAAGGGCGACTTCAGGATCCTTATGTGCACAAAGGT
GACAATGGACGACTTCCTGACAGCTCATCATGAGATGGGGCATATCCAGTATGATATG
GCATATGCTGCACAACCTTTTCTGCTAAGAAATGGAGCTAATGAAGGATTCCATGAAG
CTGTTGGGGAAATCATGTCACTTTCTGCAGCCACACCTAAGCATTTAAAATCCATTGG
TCTTCTGTCACCCGATTTTCAAGAAGACAATGAAACAGAAATAAACTTCCTGCTCAA
ACAAGCACTCACGATTGTTGGGACTCTGCCATTTACTTACATGTTAGAGAAGTGGAGG
TGGATGGTCTTTAAAGGGGAAATTCCCAAAGACCAGTGGATGAAAAAGTGGTGGGA
GATGAAGCGAGAGATAGTTGGGGTGGTGGAACCTGTGCCCCATGATGAAACATACTG
TGACCCCGCATCTCTGTTCCATGTTTCTAATGATTACTCATTCATTCGATATTACACAAG
GACCCTTTACCAATTCCAGTTTCAAGAAGCACTTTGTCAAGCAGCTAAACATGAAGG
CCCTCTGCACAAATGTGACATCTCAAACTCTACAGAAGCTGGACAGAAACTGTTCAA
TATGCTGAGGCTTGGAAAATCAGAACCCTGGACCCTAGCATTGGAAAATGTTGTAGG
AGCAAAGAACATGAATGTAAGGCCACTGCTCAACTACTTTGAGCCCTTATTTACCTGG
CTGAAAGACCAGAACAAGAATTCTTTTGTGGGATGGAGTACCGACTGGAGTCCATAT
GCAGACCAAAGCATCAAAGTGAGGATAAGCCTAAAATCAGCTCTTGGAGATAAAGCA
TATGAATGGAACGACAATGAAATGTACCTGTTCCGATCATCTGTTGCATATGCTATGAG
GCAGTACTTTTTAAAAGTAAAAAATCAGATGATTCTTTTTGGGGAGGAGGATGTGCGA
GTGGCTAATTTGAAACCAAGAATCTCCTTTAATTTCTTTGTCACTGCACCTAAAAATGT
GTCTGATATCATTCCTAGAACTGAAGTTGAAAAGGCCATCAGGATGTCCCGGAGCCGT
ATCAATGATGCTTTCCGTCTGAATGACAACAGCCTAGAGTTTCTGGGGATACAGCCAA
CACTTGGACCTCCTAACCAGCCCCCTGTTTCCATATGGCTGATTGTTTTTGGAGTTGTG
ATGGGAGTGATAGTGGTTGGCATTGTCATCCTGATCTTCACTGGGATCAGAGATCGGA
AGAAGAAAAATAAAGCAAGAAGTGGAGAAAATCCTTATGCCTCCATCGATATTAGCA
AAGGAGAAAATAATCCAGGATTCCAAAACACTGATGATGTTCAGACCTCCTTT

Each technical feature of the above-mentioned examples can be combined arbitrarily. For the sake of concise description, not all possible combinations of each technical feature in the above examples have been described. However, as long as there is no contradiction in the combination of these technical features, they should be regarded as falling within the scope described in the present description.

The above-mentioned examples only represent several embodiments of the present disclosure, and their descriptions are relatively specific and detailed, but they should not be understood as limiting the scope of the invention patent. It should be pointed out that, for those of ordinary skill in the art, without departing from the concept of the present disclosure, several modifications and improvements can also be made, and these all fall within the protection scope of the present disclosure. Therefore, the protection scope of the present disclosure shall be subject to the appended claims.

What is claimed is:

1. A method for constructing a self-amplifying RNA (saRNA) microvesicle delivery system, comprising the following steps:
(1) designing a saRNA expression vector 1 and a membrane protein expression vector 2;
(2) co-transfecting the saRNA expression vector 1 and the membrane protein expression vector 2 into a host cell;
(3) culturing the host cell and isolating to obtain a microvesicle containing saRNA;
wherein the saRNA comprises a target gene sequence;
the replicon in the saRNA expression vector 1 is an alphavirus replicon;
the microvesicle does not comprise a capsid protein.

2. The method according to claim 1, wherein the membrane protein is a viral membrane protein;
preferably, the viral membrane protein is selected from the viruses of Alphavirus, Ebola virus, Arenavirus, Henipavirus, Lyssavirus, Pneumovirus, Alpharetrovirus, Lentivirus, Vesiculovirus, Marburgvirus, Mumpsvirus, Morbillivirus, Influenzavirus A, Influenzavirus B, Influenzavirus C, Hantavirus, Flavivirus, Hepacivirus, and/or Rubivirus;
preferably, the viral membrane protein is selected from Chikungunya virus (CHIKV), Venezuelan equine encephalitis virus (VEEV), Ebola virus (EBOV), Nipah virus (NIV), Lassa virus (LASV), Rabies virus (RABV), Respiratory syncytial virus (RSV), and/or H7N1 viruses.

3. The method according to claim 1, wherein the target gene is a therapeutic gene or an antigen gene;
preferably, the therapeutic gene is an immunostimulatory gene or a suicide gene;
preferably, the antigen gene is a receptor gene, such as human angiotensin-converting enzyme II (hACE2);
preferably, the nucleotide sequence of hACE2 is as shown in SEQ ID NO: 15.

4. The method according to claim 1, wherein the host cell is a mammalian cell;
preferably, the mammalian cell is selected from a 293 cell, a baby hamster kidney 21 (BHK-21) cell, and/or a verda reno (VERO) cell.

5. A microvesicle delivery vector prepared by the method according to claim 1, wherein the delivery vector comprises a microvesicle with a viral membrane protein embedded on the surface and saRNA packaged in the vesicle, the microvesicle does not comprise a capsid protein;
preferably, the saRNA also comprises a target gene sequence.

6. The microvesicle delivery vector according to claim 5, wherein the microvesicle is a vesicular body with a bilayer membrane structure assembled from a viral envelope protein and a lipid bilayer.

7. The microvesicle delivery vector according to claim 5, wherein the particle size of the microvesicle is about 10 nm-1000 nm;
preferably, the particle size of the microvesicle is about 50-100 nm;
preferably, the particle size of the microvesicle is about 58 nm.

8. A cell stably expressing the microvesicle delivery vector according to claim 5.

9. A method of treating a disease, wherein the method comprises administering a target drug comprising the microvesicle delivery vector according to claim 5.

10. A method of treating a disease, wherein the method comprises administering a target drug comprising the cell according to claim 8.

11. A method of preventing a disease, wherein the method comprises administering a vaccine preparation comprising the microvesicle delivery vector according to claim 5.

12. A method of preventing a disease, wherein the method comprises administering a vaccine preparation comprising the cell according to claim 8.
